(19) United States
(12) Patent Application Publication
(10) Pub. No.: US 2025/0005902 A1
(43) Pub. Date: Jan. 2, 2025

(54) SYSTEMS AND METHODS FOR PROMOTING CORAL GROWTH, HEALTH, OR RESILIENCY

(71) Applicant: Coral Vita Inc., Dover, DE (US)

(72) Inventors: Alec Halpern, Freeport (BS); Samuel Teicher, Freeport (BS); Amir Matouk, Freeport (BS); Joseph Oliver, Freeport (BS); Kathryn Lesneski, Key Largo, FL (US); Samuel Wright, Freeport (BS); Veronica Cuccurullo, Freeport (BS)

(21) Appl. No.: 18/776,022

(22) Filed: Jul. 17, 2024

Related U.S. Application Data
(63) Continuation of application No. PCT/US2023/061580, filed on Jan. 30, 2023.
(60) Provisional application No. 63/305,074, filed on Jan. 31, 2022.

(51) Int. Cl. G06V 10/764; G01N 33/18; G06N 3/0464; G06V 10/82; G06V 20/05; G06V 40/10

(52) U.S. Cl. CPC G06V 10/764; G01N 33/18; G06N 3/0464; G06V 10/82; G06V 20/05; G06V 40/10; G01N 2333/43595

(57) ABSTRACT

The present disclosure provides systems and methods that may advantageously apply trained algorithms to automatically provide for coral growth, coral health, or coral resiliency, in-situ and ex-situ, for successful outplanting to a coral's native environment. In an aspect, the present disclosure provides an automated, computer-implemented method for growing resilient coral by obtaining one or more images of one or more corals and applying a machine learning-based classifier comprising a multi-class model on the one or more images to determine a resiliency of the one or more corals based at least on a plurality of coral health features and a plurality of coral environmental features.

### CROSS REFERENCE TO RELATED APPLICATIONS

The present application is a continuation application of International Application No. PCT/US2023/061580, filed Jan. 30, 2023, which claims the benefit of U.S. Provisional Application No. 63/305,074, filed Jan. 31, 2022, which is hereby incorporated by reference in its entirety.

### BACKGROUND

The state of affairs in growing diverse and resilient coral, in-situ and ex-situ, reveals different approaches for automatically fingerprinting coral, measuring coral growth, detecting adverse environmental growth conditions, providing for favorable environmental growth conditions, monitoring and assessing coral health, growing more resilient coral, optimizing and managing coral farm operations, and outplanting resilient coral back to a coral's native environment e.g., the ocean. However, some approaches may be deficient because, for example, they do not provide for all of these features, or they may not scale so that larger coral farms or similar facilities may be operated without linearly scaling human labor, or they may be unable to manage the growth of coral in an efficient manner. These approaches may be unable to effectively scale and optimize the farming of coral, and thus current reef restoration approaches may be unable to effectively and efficiently grow coral at scale for restoration. Without ecosystem-scale restoration, coral health continues to decline globally, for example, due to warming and acidifying of oceans along with other factors that threaten coral survival.

Also, some approaches do not provide automatic methods, for example, to implement these features and to act on analysis of data to automate tasks such as feeding, cleaning, cutting, or medically treating coral. Because promoting coral health can be a complex and exhaustive endeavor, automatic methods may be needed to, for example, better promote coral health in-situ, ex-situ, or in native oceanic ecosystems. Further, some approaches may not be able to implement genetic analysis at scale, for example, genotyping and phenotyping of coral, to develop more resilient coral, for outplanting back to the coral's native environment e.g., the ocean. Because these approaches may not provide for more resilient coral at scale, existing reef restoration approaches may be limited in their ability to slow and reverse the decline of coral reefs globally.

### SUMMARY

The present disclosure can address at least the above issues, for example, by using trained algorithms (e.g., computer vision models) to provide automatic determination of coral growth, coral health, or coral resiliency using a plurality of coral features and a plurality of environmental features. Also, the present disclosure, by using trained algorithms operatively coupled with sensors and controls, for example, provides automatic monitoring and adjustment of the coral's environment such as water quality. Further, the present disclosure, by using trained algorithms operatively coupled with sensors and controls, for example, can provide automatic monitoring of coral health such as bleaching of coral, exposed coral skeleton, or coral tissue loss. By the present disclosure, automatic adjustment of coral environment through assessment of coral health, by trained algorithms, for example, provides for effectively assessing the resiliency of coral that are more likely to prosper in a coral's native environment (e.g., the ocean).

The present disclosure is key for growing resilient coral in-situ and ex-situ for later outplanting to the coral's native environment such as the ocean. In-situ and ex-situ environments may include coral farms, coral nurseries, or other assisted-growth environments. Native environments may include ocean environments other than coral farms, coral nurseries, or other unassisted-growth environments. Embodiments of the present disclosure may encourage restoration and repopulation of coral reefs globally. Doing so also benefits the entire oceanic ecosystem and systems that rely on the oceanic ecosystem. For example, systems that rely on oceanic ecosystems may include fishery industries by improving marine species quantity and diversity, tourism industries by improving desirability of visiting coral reef restoration sites, local communities by improving employment, food security, or cultural heritage, or construction industries by improving erosion and flooding of coastal properties through the ability of coral reef restoration sites to attenuate waves.

An aspect of the present disclosure is an automated computer-implemented method for growing resilient corals, comprising (a) obtaining one or more images of the one or more corals and (b) applying a machine learning-based classifier comprising a multi-class model on the one or more images to determine a resiliency of the one or more corals based at least on a plurality of coral health features and a plurality of coral environmental features.

In some embodiments, the classifier analyzes the plurality of images comprising images in the visible electromagnetic spectrum. In some embodiments, the classifier analyzes the plurality of images comprising images in the infrared electromagnetic spectrum. In some embodiments, the classifier analyzes the plurality of images comprising images in the ultraviolet electromagnetic spectrum. In some embodiments, the classifier analyzes the plurality of coral health features comprising coral bleaching, coral growth, exposed coral skeleton, and coral tissue loss. In some embodiments, the classifier analyzes the plurality of coral environmental features comprising pH and salinity of water in which the one or more corals are submerged. In some embodiments, the classifier analyzes the plurality of coral environmental features comprising levels of calcium, phosphate, nitrogen, nitrate, nitrite, and dissolved oxygen in the water in which the one or more corals are submerged. In some embodiments, the classifier predicts coral resiliency for a likelihood of successful outplanting to an in situ environment such as the ocean.

Another aspect of the present disclosure is a machine learning-based classifier configured to (a) receive one or more images of one or more corals and (b) utilizing a neural network to classify the one or more corals based on the one or more images according to a plurality of coral health features and a plurality of coral environmental features.

In some embodiments, the classifier analyzes the plurality of images comprising images in the visible electromagnetic spectrum. In some embodiments, the classifier analyzes the plurality of images comprising images in the infrared electromagnetic spectrum. In some embodiments, the classifier analyzes the plurality of images comprising images in the ultraviolet electromagnetic spectrum. In some embodiments, the classifier analyzes the plurality of coral health features comprising coral bleaching, coral growth, exposed coral skeleton, and coral tissue loss. In some embodiments, the classifier analyzes the plurality of coral environmental features comprising pH and salinity of water in which the one or more corals are submerged. In some embodiments, the classifier analyzes the plurality of coral environmental features comprising levels of calcium, phosphate, nitrogen, nitrate, nitrite, and dissolved oxygen in the water in which the one or more corals are submerged. In some embodiments, the classifier predicts coral resiliency for a likelihood of successful outplanting to an in situ environment.

Another aspect of the present disclosure is an apparatus for growing resilient corals, comprising: (a) a rig wherein the rig comprises at least a top portion, a middle portion, and a bottom portion; (b) the top portion of the rig configured to comprise at least a plurality of sensors and at least a plurality of controllers; (c) the middle portion of the rig configured at least elevate the top portion of the rig above the bottom portion of the rig; and (d) the bottom portion of the rig configured to comprise at least a plurality of water tanks, a plurality of coral beds, a plurality of sensors, and a plurality of plumbing.

Another aspect of the present disclosure is a system for evaluating coral growth, coral health, or coral resiliency, comprising (a) a memory for storing a set of software instructions, and one or more processors that are configured to execute the set of software instructions to implement the method of any of claims the present disclosure and (b) a computer program product having a non-transitory computer readable medium that comprises program instructions for causing at least one processor to carry out the method of any of claims the present disclosure.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

References will be made to embodiments of the present disclosure, examples of which may be illustrated in the accompanying drawings (also “Figure” and “FIG.” herein). The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts an example embodiment (isometric view) of an environmental coral farm booth (ECoFaB) system or rig according to the present disclosure;

FIG. 2 depicts an example embodiment (orthographic view) of an environmental coral farm booth (ECoFaB) system or rig according to the present disclosure;

FIG. 3 depicts an example embodiment (orthographic view) of an environmental coral farm booth (ECoFaB) system or rig according to the present disclosure;

FIG. 4 depicts an example embodiment of a control and communications architecture of an environmental coral farm booth (ECoFaB) system or rig of the present disclosure;

FIG. 5 depicts an example embodiment of an imaging sensor in relation to an environmental coral farm booth (ECoFaB) system or rig of present disclosure;

FIG. 6 depicts an example embodiment of a coral growth bed of an environmental coral farm booth (ECoFaB) system or rig of the present disclosure;

FIG. 7 depicts an example embodiment of a plurality of coral growth beds of an environmental coral farm booth (ECoFaB) system or rig of the present disclosure;

FIG. 8 depicts an example embodiment of an enclosure of an environmental coral farm booth (ECoFaB) system or rig of the present disclosure;

FIG. 9 depicts an example embodiment of an enclosure of an environmental coral farm booth (ECoFaB) system or rig of the present disclosure;

FIG. 10 depicts a computer system that is programmed or otherwise configured to implement methods disclosed herein;

FIG. 11 depicts an example embodiment of a high-level architecture of an environmental coral farm booth (ECoFaB) system or rig of the present disclosure;

FIGS. 12A-12F depict example training, validation, and testing of a computer vision model for coral plug detection of the present disclosure;

FIGS. 13A-13D depict example training, validation, and testing of a computer vision model for coral segmentation of the present disclosure;

FIGS. 14A-14E depict example training, validation, and testing of a computer vision model for coral health of the present disclosure;

FIGS. 15A-15B depict example training, validation, and testing of a computer vision model for coral growth of the present disclosure;

FIG. 16 depicts example training, validation, and testing of a computer vision model for coral hygiene or algae growth of the present disclosure;

FIGS. 17A-17B depict example training, validation, and testing of a computer vision model for coral fingerprinting of the present disclosure;

FIGS. 18A-18C depict high-level data architectures that may be configured to implement methods of the present disclosure;

FIGS. 19A-19D depict dashboards that may be generated and configured for users or operators of the present disclosure;

FIGS. 20A-20B depict embodiments of an environmental coral farm booth (ECoFaB) system or rig.

FIGS. 21A-21E depict embodiments of an environmental coral farm booth (ECoFaB) system or rig of the present disclosure;

FIG. 22 depicts a high-level architecture of a coral life support system (LSS) having plumbing features to control injection and extraction of water and nutrients for an environmental coral farm booth (ECoFaB) system or rig of the present disclosure; and

FIG. 23. depicts an embodiment of sensors (e.g., cameras), data communications, power supplies, communications, circuitry and the like for an environmental coral farm booth (ECoFaB) system or rig of the present disclosure.

### DETAILED DESCRIPTION

While various embodiments have been shown and described herein, such embodiments are provided by way of example only. Numerous variations, changes, and substitutions may occur without departing from the scope of the disclosure. It should be understood that various alternatives to the embodiments described herein may be employed.

Various terms used throughout the present description may be read and understood as follows, unless the context indicates otherwise: “or” as used throughout is inclusive, as though written “and/or”; singular articles and pronouns as used throughout include their plural forms and vice versa; similarly, gendered pronouns include their counterpart pronouns so that pronouns should not be understood as limiting anything described herein to use, implement, perform, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 0.0001 milliseconds (ms), 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

Any of the algorithms described herein may be used, either individually, or combined/fused into one or more algorithms that are each configured or capable of performing a plurality of tasks. Accordingly, an algorithm as described herein may be configured for a specific task or may be configured for multiple tasks. An algorithm can be a single discrete algorithm. Alternatively, an algorithm may comprise a plurality or series of algorithms that work in tandem to perform multiple tasks, either concurrently, sequentially, or in any specific order. Depending on the tasks to be performed, the algorithms described herein can be modified or combined in multiple different ways and can be tailored for a variety of different tasks or applications to optimize coral farming. Coral can generally include coral, coral fragments, coral microfragments, coral broodstock, and the like.

Recognized herein is the need for systems and methods for improving coral growth, coral health, or coral resiliency in-situ and ex-situ, using machine learning techniques, that provide for scalable and effective growth of coral for outplanting resilient coral to a native environment such as the ocean. The present disclosure, using trained algorithms, improves over the ability of human labor to promote coral growth, coral health, or coral resiliency.

The present disclosure relates generally to growing coral in-situ and ex-situ, and then outplanting coral to a native environment such as the ocean. More specifically, the present disclosure provides systems and methods, via trained algorithms, for detecting coral plugs, segmenting coral, determining or predicting coral health, determining or predicting coral growth, determining or predicting coral hygiene, fingerprinting coral, determining or predicting adverse environmental growth conditions, providing for favorable environmental growth conditions, determining or predicting coral resiliency, growing more resilient coral, and outplanting resilient coral back to an in situ environment such as the ocean.

The present disclosure, by trained algorithms, can also support operations of in-situ coral farms and ex-situ coral farms, through automatic and real-time analysis of a plurality of data. Traditional coral farming approaches may require extensive human labor that is susceptible to error and without standardized analysis. For example, a trained coral farmer may require at least about 60 minutes to assess the health of at most about 10,000 corals and with significant levels of error. For example, a trained coral farmer can assess the health of 10,000 coral fragments over the course of an hour, but this trained coral farmer may only be able to assess a threshold level of coral hygiene (e.g., algae growth), a threshold level of coral health indications, a threshold level of a plurality of parasites, or a threshold level of a plurality of disease vectors. During this hour, the trained coral farmer may also misdiagnose coral health conditions of at least about 10-20% of the coral. The present disclosure, by trained algorithms, improves upon current approaches by assessing coral growth, coral health, or coral resiliency earlier than threshold levels of a trained coral farmer, of at least about 30,000 corals in less than about 60 minutes with lower error levels. Further, the present disclosure, by trained algorithms, improves upon current approaches by assessing coral environmental conditions earlier than threshold levels of a trained coral farmer. Trained algorithms may include, for example, computer vision models. Computer vision models may use images of coral at other than optical wavelengths for more detailed analysis of coral than can be achieved by humans.

For example, the present disclosure, by trained algorithms, may use dynamic, real-time control to modulate the plurality of coral growth, health, or resiliency features and the plurality of coral environmental features in order to promote coral growth, coral health, or coral resiliency. For example, the present disclosure, may use a closed-loop feedback architecture wherein trained algorithms collect and analyze the plurality of data from the plurality of sensors to modulate, in real-time or in delayed time, the plurality of coral growth, health, or resiliency features and the plurality of coral environmental features. For example, trained algorithms may modulate the plurality of coral growth, health, or resiliency features and the plurality of coral environmental features based on predictions of coral growth, coral health, or coral resiliency over a time period or intermediate time periods within the time period.

For example, the present disclosure, by trained algorithms may generate predictions based on the plurality of current data from the plurality of sensor data. The trained algorithms may generate predictions based on the plurality of historical data from the plurality of sensor data. The trained algorithms may generate predictions based on the plurality of both current data and historical data from the plurality of sensor data. The trained algorithms may generate predictions based on comparing the plurality of current data to the plurality of historical data from the plurality of sensor data. The trained algorithms may generate predictions, for example, using a plurality of virtual simulations with a plurality of input variables. The plurality of input variables may comprise local variables. The local variables may comprise, for example, pH of the water tank in which the coral are submerged. The plurality of input variables may comprise global variables. The global variables may comprise, for example, ordinary weather patterns such as temperature, rain, wind, and pressure. The global variables may comprise, for example, extraordinary weather patterns such as hurricanes or typhoons. The plurality of input variables may comprise both local and global variables.

Promoting coral growth, coral health, or coral resiliency can comprise assisted evolution by the trained algorithms of the present disclosure. Assisted evolution can be associated with methods of genetic sequencing or phenotyping of coral. Algorithms may be trained, tested, or validated, in part, using genetic data generated or received from genetic sequencing of coral. Algorithms may be trained, tested, or validated, in part, using phenotype data generated or received from phenotyping of coral. Assisted evolution, by the trained algorithms, may include stress hardening. Stress hardening may comprise subjecting coral to adverse growth or environmental conditions (e.g., adverse temperature, pH, salinity, acidification, nutrient loads, flow rates, and the like) that threaten coral growth, coral health, or coral resiliency in repeated short-term periods. After a number of rounds through exposure to adverse growth or environmental conditions, coral may build up a tolerance to adverse growth or environmental conditions. Adverse conditions may comprise, for example, adverse temperature or flow conditions of the water tank in which the coral are submerged, adverse acidification or salinity of the water tank in which the coral are submerged, or adverse nutrient conditions of the water tank in which the coral are submerged. Assisted evolution may further include genetic selection or breeding. Genetic selection or breeding can comprise selecting, by the trained algorithms, the most resilient coral, fragmenting the most resilient coral to create a new batch of coral that are clones of the parent coral, and sexually breeding the fragmented coral to create a new generation of coral genotypes, and repeating again to select the most resilient coral for outplanting to an in situ environment such as the ocean. In some cases, clones can be returned directly back to the ocean without sexually breeding them. Assisted evolution, by the trained algorithms, may also determine or predict which coral types or species are optimal (e.g., genetically fit coral) for different growth or environmental conditions.

The automated environmental coral farm booth (ECoFaB) system or rig of the present disclosure, by the trained algorithms, can allow for detecting coral plugs, segmenting coral, determining or predicting coral health, determining or predicting coral growth, determining or predicting coral hygiene, fingerprinting coral, determining or predicting adverse environmental growth conditions, providing for favorable environmental growth conditions, determining or predicting coral resiliency, growing more resilient coral, or outplanting resilient coral back to an in situ environment such as the ocean. The ECoFaB system may comprise at least a physical rig for growing coral, housing sensors and controllers, or containing growth medium; sensors operatively coupled to the rig for monitoring, by the trained algorithms, a plurality of coral growth, health, or resiliency indications; sensors operatively coupled to the rig for monitoring, by the trained algorithms, a plurality of coral environmental conditions; trained algorithms for analyzing the plurality of sensor data and controlling the plurality of environmental conditions to promote coral growth, coral health, or coral resiliency; an automatic cleaning apparatus for cleaning coral tanks, coral trays, coral plugs, or coral determined or predicted to be dirty by the trained algorithms; a coral treatment apparatus for medically treating the coral determined or predicted to be unhealthy by the trained algorithms; coral life support systems (LSS) associated with plumbing features; or a collection of coral tanks, coral trays, coral plugs, and coral.

In some cases, the term “ECoFaB system” can be used interchangeably with the term “ECoFaB rig.” For example, the ECoFaB system or the ECoFaB rig can include features such as a rig, coral tanks, coral trays, coral plugs, coral, coral growth environment, plumbing features, coral environmental features, and coral life support systems (LSS). In some cases, the ECoFaB rig can be separate from and operatively coupled to the ECoFaB system. In some cases, the ECoFaB system comprises the ECoFaB rig.

### ECoFaB System or Rig

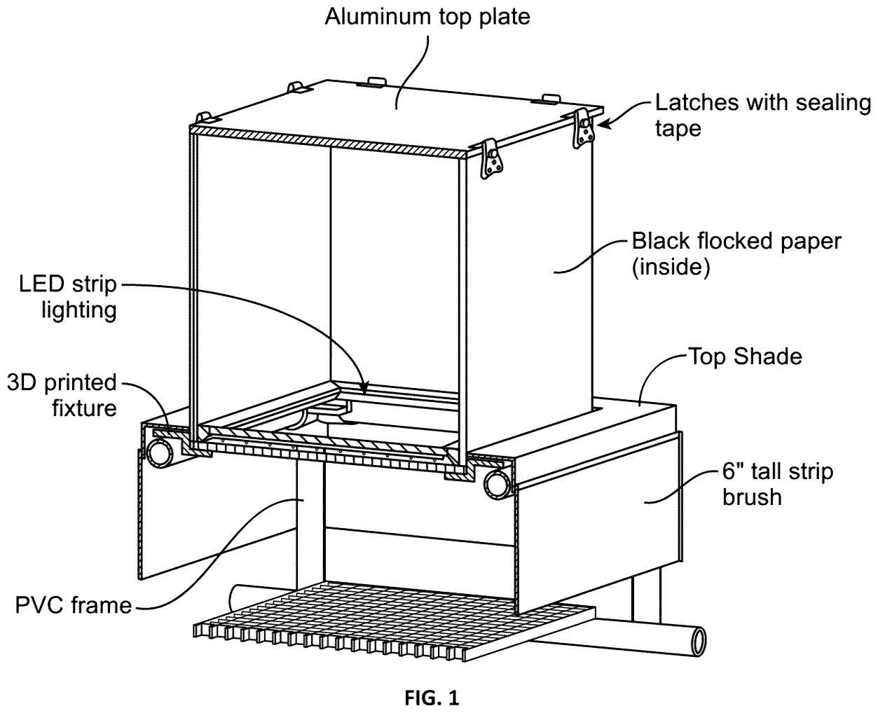

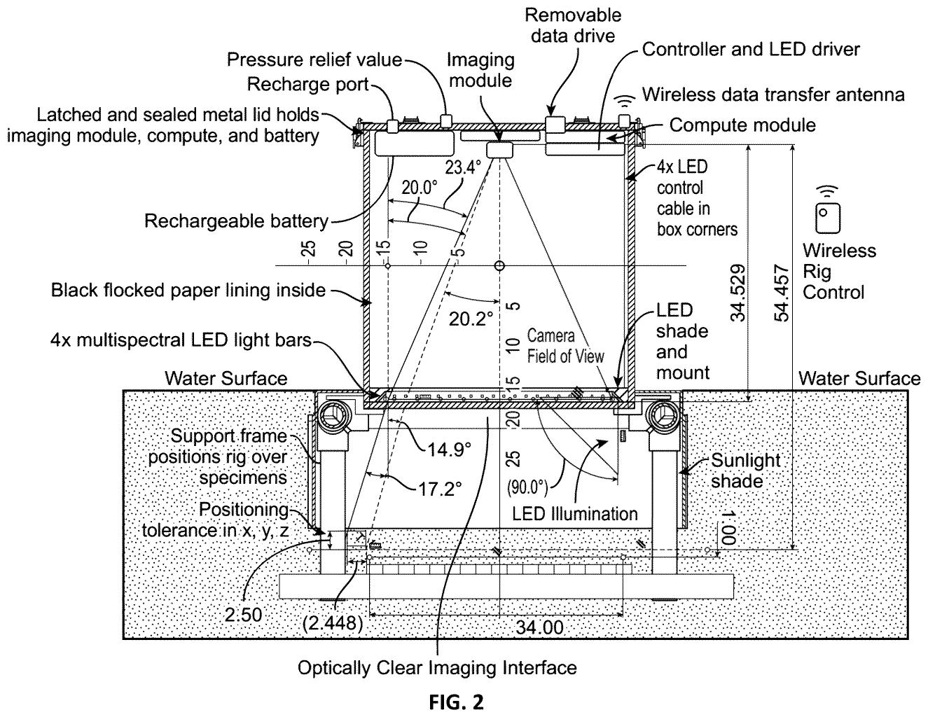

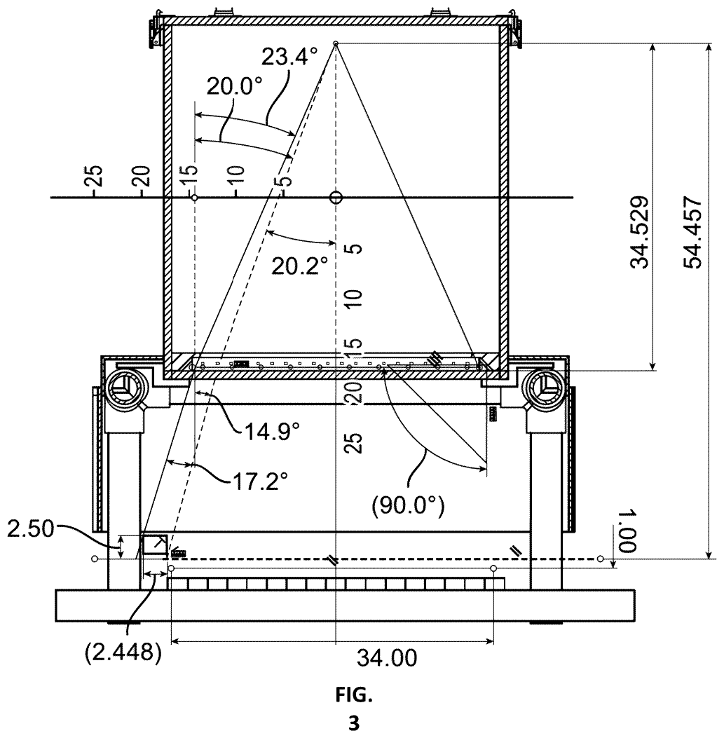

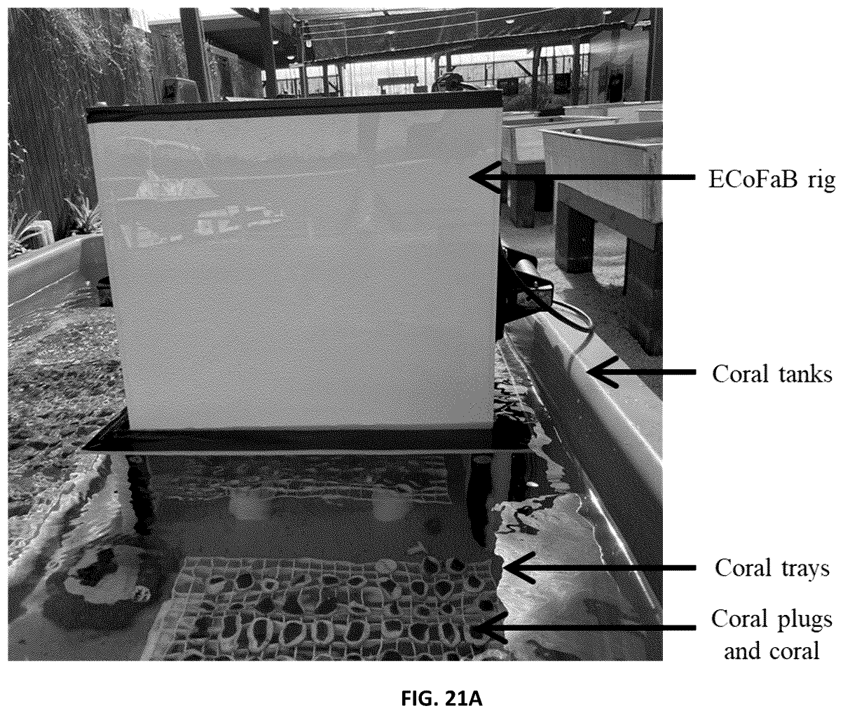

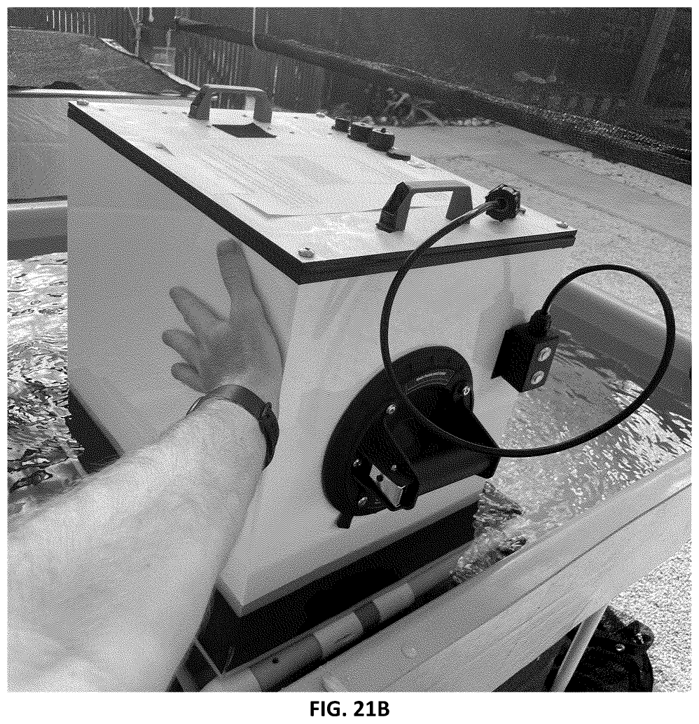

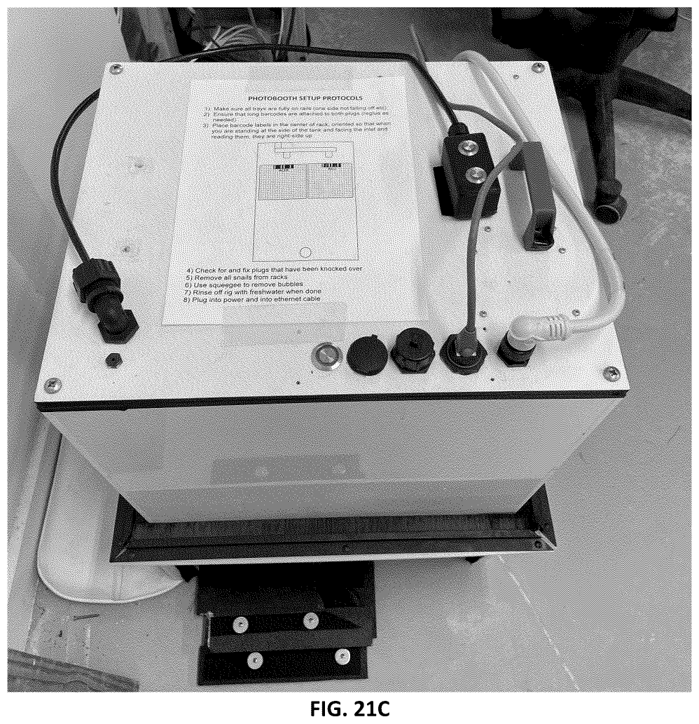

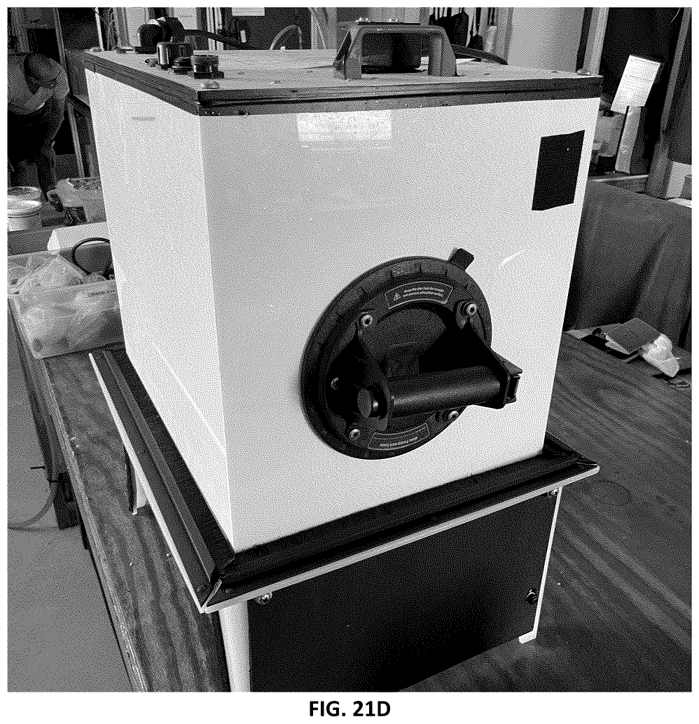

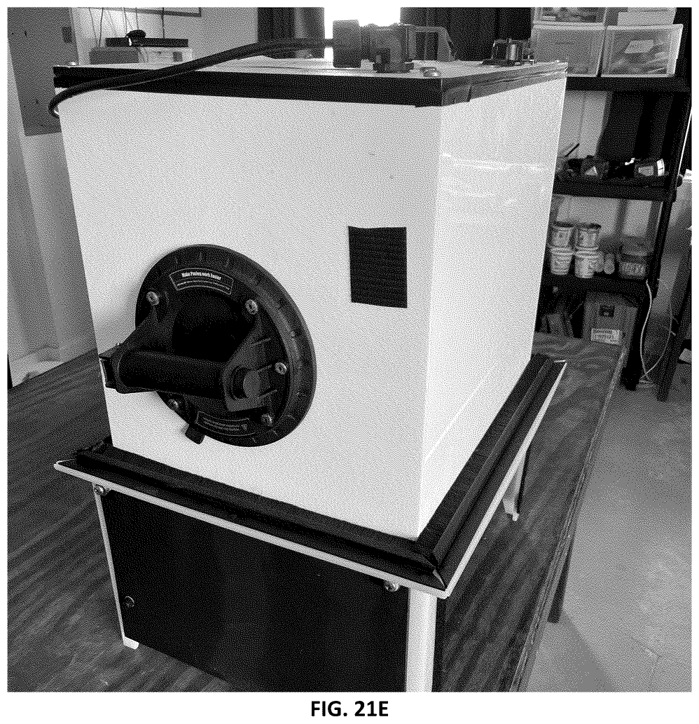

As an example of an ECoFaB system, FIGS. 1 and 21A-21E show a plurality of features wherein the ECoFaB system or rig, for example, may be operatively coupled to coral tanks, coral trays, coral plugs, coral, coral growth environment, plumbing features, coral environmental features, and coral life support systems (LSS). The ECoFaB system or rig can comprise a top portion (“booth”), a middle portion (“support”), and a bottom portion (“tank”). The top portion may comprise five connected sides of a box with, for example, dimensions shown in FIGS. 2 and 3. The bottom sixth side of the box, further connected to the five connected sides as a box, can comprise a transparent fabrication material for simultaneous viewing of the middle portion or the bottom portion from the top portion. The five connected sides of the top portion may be fabricated from any suitable material providing optical opaqueness to block some or all ambient light, rigidity, or protection from ambient conditions. For example, the five connected sides of the top portion may be fabricated from aluminum. The bottom sixth side may be fabricated from any suitable material providing optical transparency to allow optical viewing from above into the middle portion or the bottom portion, rigidity, or protection from ambient conditions. For example, the bottom sixth side may be fabricated from optically transparent glass or plastic. The five sides of the top portion may include a material lining the inside of the top portion for preventing some or all light from entering the booth as may be required for performance of the sensors (e.g., cameras). For example, the top, left, right, front, and back sides of the five sides of the top portion may include black flocked paper. The top side of the top portion may include a means for quickly releasing or removing the top side for easier access to the inside of the top portion, middle portion, or bottom portion. For example, the top side may include latches with sealing tape connected to the left, right, front, or back sides of the top portion. Alternatively, the top side may be hingedly connected to any one of the left, right, front, or back sides. The rig may contain handles for lifting, moving, rotating, or positioning the rig.

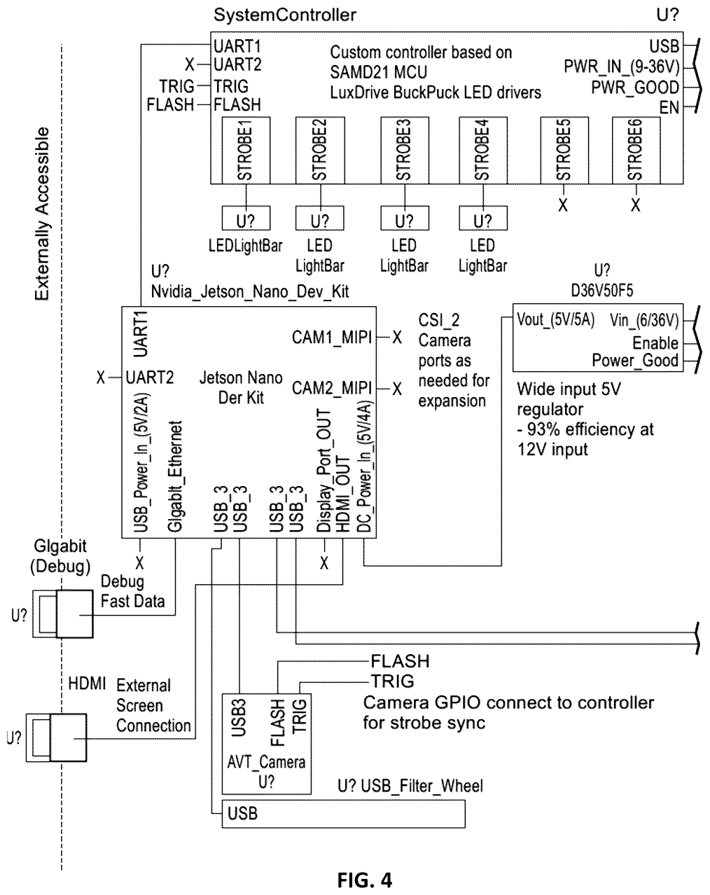

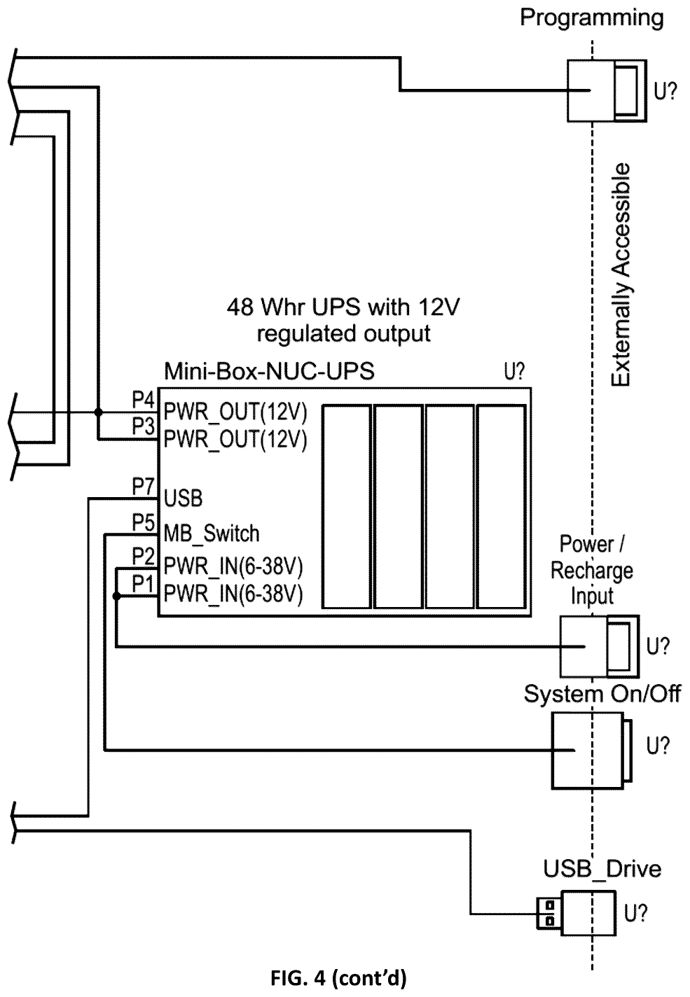

The top portion can further comprise connections for a plurality of features as shown, for example, in FIGS. 2, 3, and 23. The features in the top portion may comprise a plurality of sensors such as, for example, a plurality of imaging sensors (e.g., optical cameras, UV cameras, or IR cameras) for imaging the coral or for imaging environmental conditions. The plurality of imaging sensors (described elsewhere herein) may provide, for example, imaging coral at different wavelengths of the electromagnetic spectrum to provide indications of coral growth, coral health, or coral resiliency. The plurality of imaging sensors may provide, for example, imaging coral at different wavelengths of the electromagnetic spectrum to provide a plurality of biological or physical data about coral. The features in the top portion may comprise a plurality of memory devices for storing data from the plurality of sensors. The features in the top portion may comprise a plurality of computing devices for controlling automatic operation of the ECoFaB system. For example, a computing device may be a programmable logic controller (PLC) as shown in FIG. 4 and described elsewhere herein. The features in the top portion may comprise a plurality of power supplies for providing power to the plurality of features in the top portion. For example, the power supply may comprise a plurality of rechargeable batteries that are recharged from a plurality of recharging ports in the top portion. The features in the top portion may comprise a plurality of features for controlling the pressure in the ECoFab system. For example, the ECoFab system may use a plurality of programmable pressure relief valves to control the pressure in the booth. The features in the top portion may comprise a plurality of light sources for illuminating the inside of the ECoFab system. For example, the top portion may comprise multispectral LED light sources. The features in the top portion may comprise a plurality of communication or control cables for communication or control between the plurality of features in the top portion. For example, the top portion may comprise a controller, driver, or cable for operation of the multispectral LED light sources. The features in the top portion may comprise a plurality of communication or wireless antennas. For example, the top portion may include a wireless antenna for communicating the plurality of sensor data to a data server (described elsewhere herein) for further analysis, by the trained algorithms, of coral growth, coral health, coral resiliency, or coral environmental conditions.

The middle portion (“support”) of the ECoFaB system or rig can comprise a plurality of features as shown, for example, in FIGS. 1 and 2. The plurality of features may comprise a support frame to elevate the top portion of the rig above the bottom portion of the rig. The support frame can also provide support for the plurality of coral tanks or the plurality of coral trays located in the bottom portion of the rig. The support frame may be fabricated from any suitable material providing structural rigidity or protection from ambient conditions. For example, the support frame may be fabricated from tubular polyvinyl chloride (PVC). The plurality of features may further comprise a means to block out ambient light similar to the top portion. For example, the middle portion may use black flocked paper.

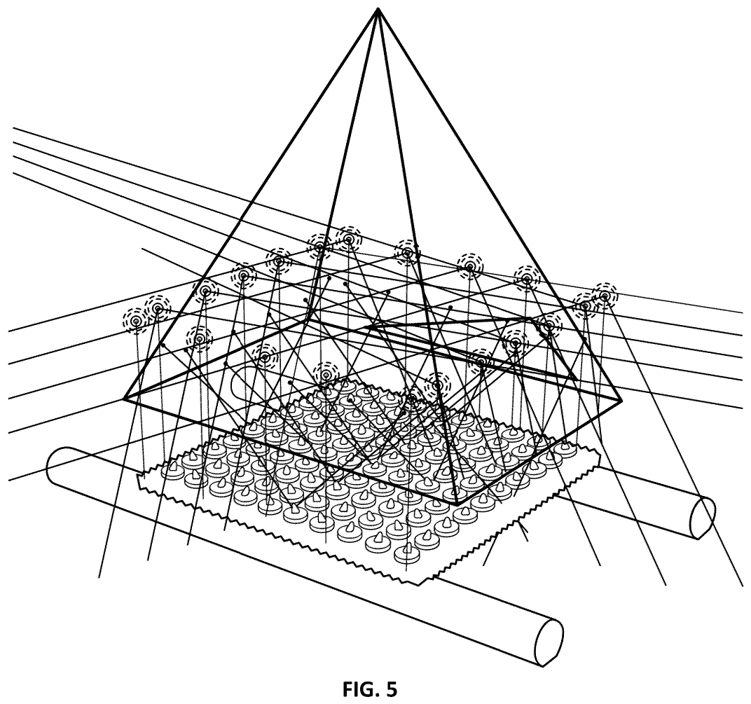

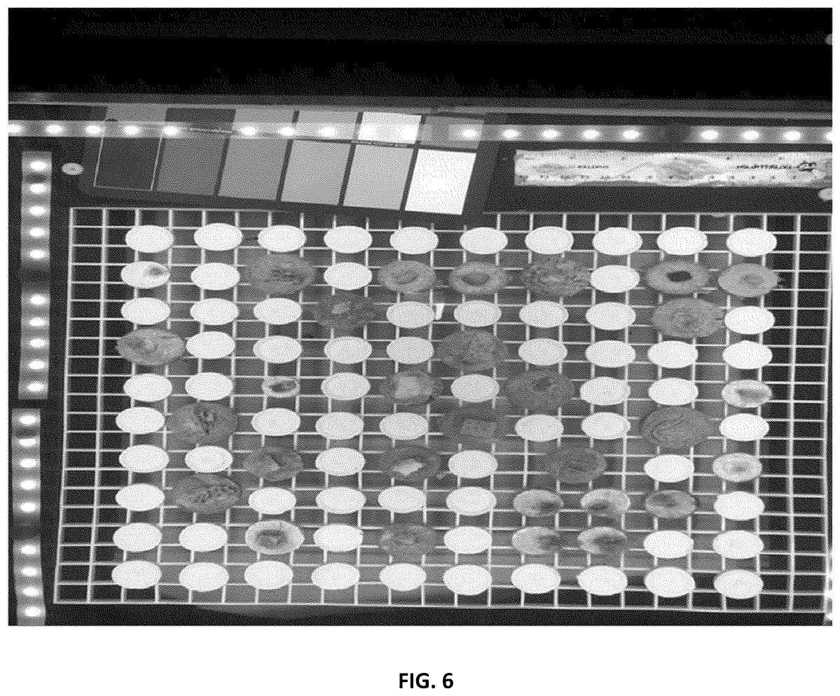

The bottom portion (“tank”) of the ECoFaB system or rig may comprise a plurality of features as shown in, for example, FIGS. 1 and 2. The plurality of features may comprise a plurality of coral trays submerged in a plurality of water tanks below the middle portion of the rig as shown in FIGS. 5, 7, and 21A-21B. The plurality of coral trays may comprise a plurality of coral growth sites (e.g., coral plugs). The plurality of coral plugs may comprise a plurality of surfaces for placing coral to optimize, by the trained algorithms, coral growth, coral health, or coral resiliency. The plurality of coral plugs on a coral tray for growing coral may comprise at least about 1, 10, 20, 30, 40, 50, 60, 70, 80, 90, 100, or more coral plugs. The plurality of coral plugs on a coral tray for growing coral may comprise at most about 100, 90, 80, 70, 60, 50, 40, 30, 20, 10 or 1 coral plugs.

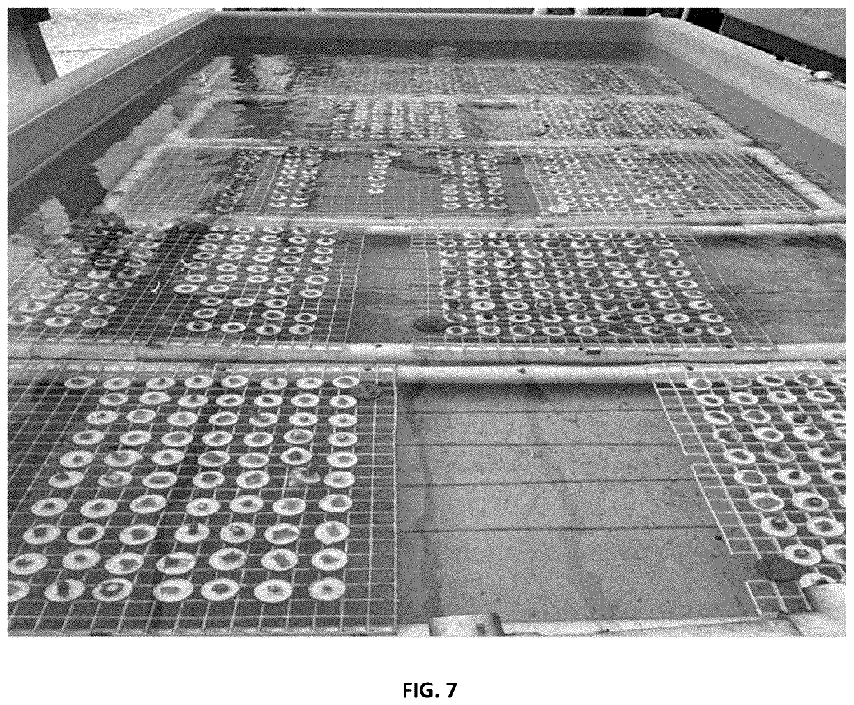

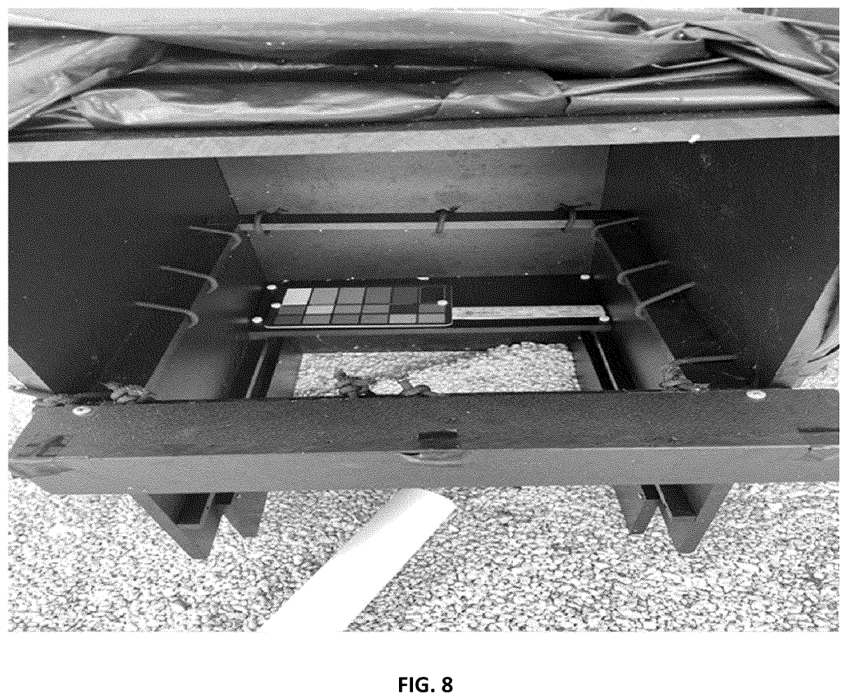

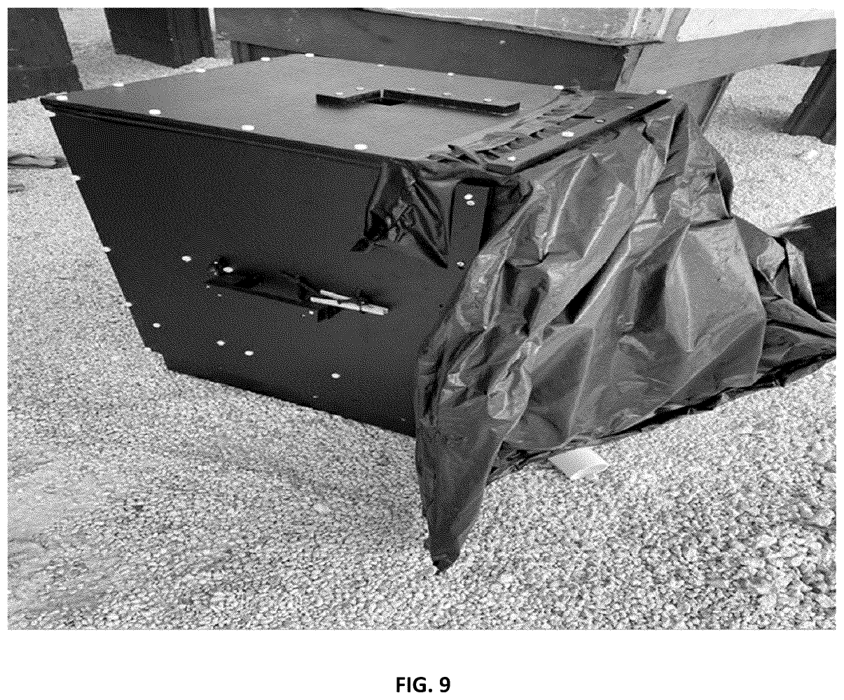

Each of the plurality of coral plugs may comprise a plurality of coral or plurality of microfragments of coral. The plurality of coral or microfragments of coral per coral plug may comprise at least about 1, 2, 3, 4, 5, 6, 7, 8, 9, 10, or more corals or microfragments of corals. The plurality of coral or microfragments of coral per coral plug may comprise at most about 10, 9, 8, 7, 6, 5, 4, 3, 2, 1, or less corals or microfragments of corals. Through the plurality of coral trays in the plurality of water tanks as shown in FIG. 7, the plurality of coral plugs or the plurality of coral may be scaled at least about 2×, 3×, 4×, 5×, 30×, or more. The plurality of coral plugs or the plurality of coral may be scaled at most about 30×, 5×, 4×, 3×, 2×, or less.

The features of the bottom portion of the system or rig further can comprise a plurality of plumbing features to operatively function as a coral life support system (LSS) to control injection and extraction of water and nutrients, by the trained algorithms, to and from the plurality of coral tanks, the plurality of coral trays, the plurality of coral plugs, the plurality of coral or microfragments of coral to affect the plurality of coral environmental conditions. The plumbing features further control injection and extraction of chemicals, by the trained algorithms, to maintain optimal environmental conditions in the water tanks for coral growth, coral health, or coral resiliency. For example, the present disclosure may inject and extract chemicals to affect water quality through adjustment of the pH and the salinity of the water tanks in which the plurality of coral are submerged. For example, optimal adjustment of the pH or salinity of the water tank may affect the growth, health, or resiliency of the coral. For example, the present disclosure may maintain a pH between about 7.8 and about 8.5 for coral growth, coral health, or coral resiliency. For example, the present disclosure may determine or predict an optimum pH for coral growth, coral health, or coral resiliency by the trained algorithms. For example, the present disclosure may maintain a salinity between about 28 parts per thousand and about 42 parts per thousand for coral growth, coral health, or coral resiliency. For example, the present disclosure may determine or predict an optimum salinity for coral growth, coral health, or coral resiliency by the trained algorithms.

The plumbing features may further control injection and extraction of a plurality of minerals, by the trained algorithms, to maintain optimal calcium for coral growth, coral growth, or coral resiliency. For example, the present disclosure may maintain a calcium level between about 360 mg/L and 450 mg/L for coral growth, coral health, or coral resiliency. For example, the present disclosure may determine or predict an optimum calcium level for coral growth, coral health, or coral resiliency by the trained algorithms. For example, the present disclosure may maintain a phosphate level between about 0.005 mg/L and about 0.1 mg/L for coral growth, coal health, or coral resiliency. For example, the present disclosure may determine or predict an optimum phosphate level for coral growth, coral health, or coral resiliency by the trained algorithms. For example, the present disclosure may maintain a nitrogen level between about 0.001 mg/L and about 0.05 mg/L for coral growth, coral health, or coral resiliency. For example, the present disclosure may determine or predict an optimum nitrogen level for coral growth, coral health, or coral resiliency by the trained algorithms. For example, the present disclosure may maintain a nitrate level between about 0 mg/L and 10 mg/L for coral growth, coral health, or coral resiliency. For example, the present disclosure may determine or predict an optimum nitrate level for coral growth, coral health, or coral resiliency by the trained algorithms. For example, the present disclosure may maintain a nitrite level of about 0 mg/L. For example, the present disclosure may maintain a dissolved oxygen level between about 6.5 mg/L and about 8 mg/L for coral growth, coral health, or coral resiliency. For example, the present disclosure may determine or predict an optimum dissolved oxygen level for coral growth, coral health, or coral resiliency by the trained algorithms.

The plumbing features may further control injection of extraction of water in the coral tanks, by the trained algorithms, at a plurality of different temperatures in which the plurality of coral are submerged. For example, optimal adjustment of the coral tank water temperature may improve the growth, health, or resiliency of the coral. For example, the present disclosure may maintain a coral tank water temperature between about 18° Celsius (about 64° Fahrenheit) and about 40° Celsius (about 104° Fahrenheit). For example, the present disclosure may determine or predict an optimum temperature for coral growth, coral health, or coral resiliency based on analysis of coral health, coral growth, or coral resiliency by the trained algorithms. The plumbing features further control injection and extraction of water in the coral tanks, by the trained algorithms, at a plurality of flow rates in which the plurality of coral are submerged. For example, optimal adjustment of the flow rates may improve the growth, health, or resiliency of the coral. For example, the present disclosure may maintain a flow rate between about 5 cm/s and about 15 cm/s. For example, the present disclosure may determine or predict an optimum flow rate for coral growth, coral health, or coral resiliency based on analysis of coral growth, coral health, or coral resiliency by the trained algorithms.

The features of the bottom portion of the system or rig may further comprise a plurality of sensors to detect or control optimum conditions for coral growth, coral health, or coral resiliency. For example, the plurality of sensors can detect water quality such as pH or salinity in which the plurality of coral are submerged. For example, the sensors may detect the temperature of the coral tank water in which the plurality of coral are submerged. For example, the sensors can detect flow rate of the water tank in which the plurality of coral are submerged. For example, the sensors can detect mineral levels of the coral tank water in which the plurality of coral are submerged. For example, the minerals may comprise calcium, phosphate, nitrogen, nitrate, nitrite, or dissolved oxygen. The present disclosure, by the trained algorithms, analyzes sensor data to determine or predict optimum conditions for coral growth, coral health, or coral resiliency described elsewhere herein.

The features of the three portions of the system or rig further may comprise a plurality of sensors to detect or control a plurality of coral environmental conditions, a plurality of coral health conditions, a plurality of coral growth conditions, or a plurality of coral resiliency conditions. The plurality of conditions may comprise light, algae, competition for growth space, zooxanthellae productivity (“photosynthesis rate”), density, or substrate composition.

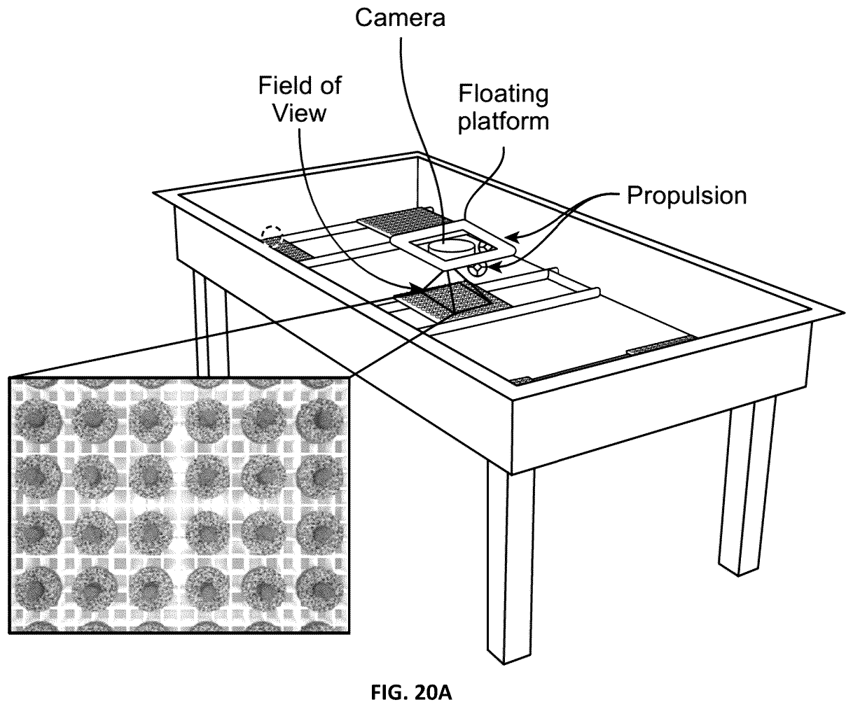

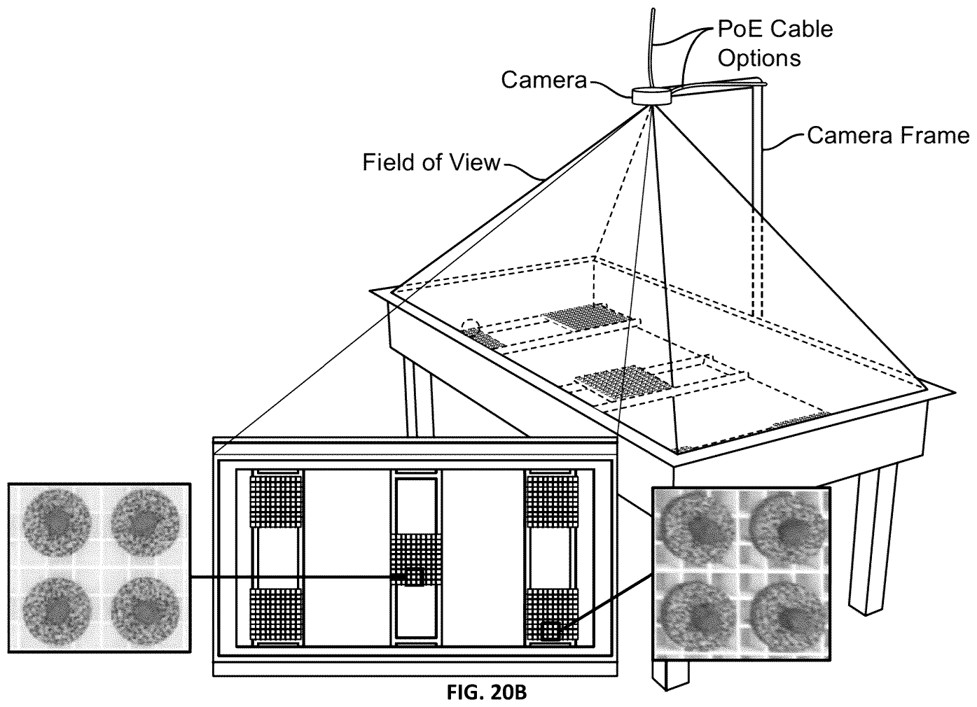

In another embodiment of an ECoFaB system, FIG. 20A shows a plurality of features wherein the rig may be operatively coupled to coral tanks, coral trays, coral plugs, coral, or coral environmental conditions. For example, the rig may float on or over a surface of water contained within a coral tank to generate images of coral trays, coral plugs, coral, or coral environmental conditions. A floating rig design can be lighter and more easily maneuvered over coral trays, coral plugs, or coral compared to a fixed rig design. A floating rig design can float over coral trays, coral plugs, or coral and be configured with a plurality of sensors or a plurality of cameras, described elsewhere herein, to capture multiple views or perspectives to generate 3D models of coral plugs or coral. Multiple views or perspectives of coral trays, coral plugs, or coral can improve the accuracy of the trained algorithms (e.g., computer vision models described elsewhere herein) to improve coral growth, coral health, or coral resiliency. The multiple cameras may be configured to rotate around coral trays, coral plugs or coral.

In another embodiment of an ECoFaB system, FIG. 20B shows a plurality of features wherein the rig may be operatively coupled to coral tanks, coral trays, coral plugs, coral or coral growth environment. For example, the rig may be positioned over an entire coral tank to generate images of coral trays, coral plugs, coral, or coral environmental conditions. In another embodiment of an ECoFaB system, the rig can be manufactured from lightweight materials such as carbon fiber or plastic. Sensors, batteries, computing devices, communication devices, circuitry and the like, described elsewhere herein, can be mounted in a backpack style wearable device and operatively coupled to one or more handheld imaging devices. The handheld imaging devices can be waterproof to provide for imaging coral trays, coral plugs, or coral beneath a surface of water in a coral tank. In another embodiment of an ECoFaB system, the rig may be further configured with a range extending automated coral husbandry (REACH) system. The REACH system may comprise a robotic range extending tool with an ability to access coral tanks, coral trays, coral plugs, or coral. The REACH system may locate itself within the coral environment using a combination of trained algorithms or pre-defined movements. The REACH system may be configured with multiple payload sections having connections for power, data, or communications. Payload sections may include sensors, imaging devices, fluid dispensing systems for coral feeding or disease treatment, or environmental sensors.

### ECoFaB Sensors

The trained algorithms analyze a plurality of data from a plurality of sensors to automatically optimize coral growth, coral health, or coral resiliency. For example, coral health may be determined or predicted, via the trained algorithms, by imaging coral at different wavelengths. The plurality of imaging sensors (e.g., multispectral or hyperspectral cameras) for imaging coral can comprise optical imaging sensors for imaging coral in the visible spectrum of the electromagnetic spectrum (about 400 nm to about 700 nm), infrared (IR) imaging sensors for imaging coral in the infrared spectrum of the electromagnetic spectrum (about 780 nm to about 1 mm), or ultraviolet (UV) imaging sensors for imaging coral in the ultraviolet spectrum of the electromagnetic spectrum (about 100 nm to about 400 nm), and other imaging sensors for imaging in other spectrums of the electromagnetic spectrum.

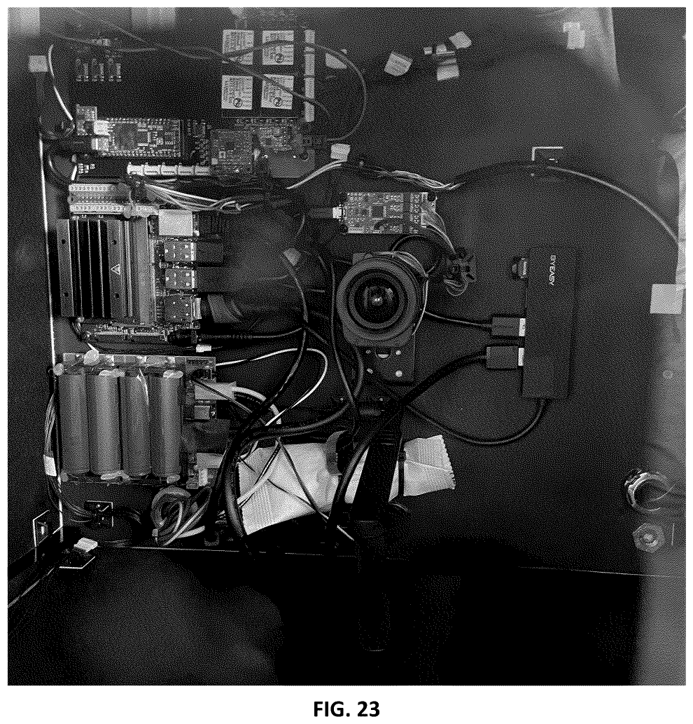

The plurality of imaging sensors, used by the trained algorithms for imaging, may comprise a single imaging sensor, for example, as shown in FIGS. 5 and 23. Alternatively, the plurality of imaging sensors may comprise multiple imaging sensors (not shown for clarity). In some cases, the plurality of sensors for imaging coral may comprise imaging sensors for a plurality of visible wavelengths, a plurality of infrared (IR) wavelengths, and a plurality of ultraviolet (UV) wavelengths. The one or more imaging sensors for visible wavelengths may include at least about 1, 2, 3, 4, 5, 6, 7, 8, 9, 10, or more sensors. The one or more imaging sensors for visible wavelengths may include at most about 10, 9, 8, 7, 6, 5, 4, 3, 2, or 1 imaging sensors. The one or more imaging sensors for IR wavelengths may include at least about 1, 2, 3, 4, 5, 6, 7, 8, 9, 10, or more imaging sensors. The one or more imaging sensors for IR wavelengths may include at most about 10, 9, 8, 7, 6, 5, 4, 3, 2, or 1 imaging sensors. The one or more imaging sensors for UV wavelengths may include at least about 1, 2, 3, 4, 5, 6, 7, 8, 9, 10, or more imaging sensors. The one or more imaging sensors for UV wavelengths may include at most about 10, 9, 8, 7, 6, 5, 4, 3, 2, or 1 imaging sensors. Examples of the imaging sensors may include, but are not limited to, charge coupled devices (CCD), metal oxide semiconductors (MOS) (e.g., complementary MOS or CMOS), modifications thereof, or functional variants thereof.

Various wavelengths may be selected, by the trained algorithms, based on a plurality of desired coral growth features, coral health features, or coral resiliency features. Features may include, for example, coloration of the coral, clarity of live tissue consistency, and growth rates. For example, the plurality of data from the plurality of visible imaging sensors may comprise bleaching of the coral as an indication of coral growth, coral health, or coral resiliency. Visible imaging may further comprise imaging of coral hygiene (e.g., algae nearby, on, or within coral plugs or coral) as an indication of coral growth, or resiliency. For example, imaging of algae on a coral plug adjacent to coral may impede coral growth. Visible imaging may further comprise imaging exposed coral skeleton or coral tissue loss as an indication of coral growth, coral health, or coral resiliency. For example, the plurality of data from the plurality of IR imaging sensors may comprise chlorophyll fluorescence as an indication of coral growth, coral health, or coral resiliency. IR imaging may further comprise detecting earlier onset of coral bleaching than visible imaging alone. For example, the plurality of data from the plurality of UV imaging sensors may comprise protein fluorescence as an indication of coral growth, coral health, or coral resiliency.

The plurality of imaging sensors may be disposable and configured for single use in a coral imaging event. Alternatively, the imaging sensor may be configured to be reusable for a plurality of coral imaging events. The plurality of coral imaging events may be for the same coral or for a plurality of different coral. The plurality of coral imaging events may be for a single coral tray or for a plurality of coral trays. The plurality of coral imaging events may be for a single coral plug or for a plurality of coral plugs. The plurality of coral imaging events may be for a single coral or coral microfragment or for a plurality of coral or coral microfragments. The imaging sensor may be reusable for at least about 2, 3, 4, 5, 6, 7, 8, 9, 10, 20, 30, 40, 50, 60, 70, 80, 90, 100, 200, 300, 400, 500, 1,000, 10,000 or more coral imaging events. The imaging sensor may be reusable for at most about 10,000, 1,000, 500, 400, 300, 200, 100, 90, 80, 70, 60, 50, 40, 30, 20, 10, 9, 8, 7, 6, 5, 4, 3, or 1 coral imaging events.

The plurality of imaging sensors may be configured to receive light signals from coral for analysis of the coral. Light signals may be reflected or emitted, for example, in the visible spectrum. Light signals may be reflected or emitted, for example, in the IR spectrum. Light signals may be reflected or emitted, for example, in the UV spectrum. The imaging sensors may be configured to detect the light signals reflected or emitted from the coral. The generated images may be one-dimensional or multi-dimensional (e.g., two-dimensional or three-dimensional). The generated images may be hyperspectral or multispectral. The generated images may include microscopic features of cellular functioning. The imaging sensors may be operatively coupled to processors. In such case, the imaging sensors may be configured to detect the light signals reflected or emitted from the coral and to convert the detected light signals into digital signals. The imaging sensors may further be configured to transmit the digital signals to processors that are capable of generating images indicative of coral growth, coral health, or coral resiliency. The trained algorithms may then analyze the images to determine coral growth, coral health, or coral resiliency and automatically adjust coral environmental features to optimize coral growth, coral health, or coral resiliency.

The plurality of imaging sensors may be associated with a plurality of cameras. The imaging sensors or cameras may have different optical axes. An optical axis of an imaging sensor and an optical axis of a camera may intersect at an angle of at least about 1 degree, 2 degrees, 3 degrees, 4 degrees, 5 degrees, 6 degrees, 7 degrees, 8 degrees, 9 degrees, 10 degrees, 20 degrees, 30 degrees, 40 degrees, 50 degrees, 60 degrees, 70 degrees, 80 degrees, 90 degrees, or more. The optical axis of the imaging sensor and the optical axis of the camera may intersect at an angle of at most about 90 degrees, 80 degrees, 70 degrees, 60 degrees, 50 degrees, 40 degrees, 30 degrees, 20 degrees, 10 degrees, 9 degrees, 8 degrees, 7 degrees, 6 degrees, 5 degrees, 4 degrees, 3 degrees, 2 degrees, 1 degree, or less. In an example, the optical axis of the imaging sensor may be orthogonal to the optical axis of the camera. Alternatively, the imaging sensor and the camera may have parallel but different longitudinal optical axes.

In some cases, the plurality of imaging sensors and the plurality of associated cameras may comprise an optics assembly with a beam splitter. The beam splitter may be configured to receive light signals, reflected or emitted, from the coral and (i) reflect the first portion of light signals in a first electromagnetic spectral range toward the imaging sensor and (ii) permit the second portion of light signals in a second electromagnetic spectral range to pass through toward the camera. Alternatively, the beam splitter may be configured to receive light signals, reflected or emitted, from the coral and (i) reflect the second portion of light signals in the second electromagnetic spectral range toward the camera and (ii) permit the first portion of light signals in the first electromagnetic spectral range to pass through toward the imaging sensor. Examples of the beam splitter may include, but are not limited to, a half mirror, a dichroic beam splitter (e.g., a shortpass or longpass dichroic mirror), or a multi-band beam splitter. In an example, the beam splitter may be a cube comprising two prisms (e.g., two triangular glass prisms) disposed adjacent to each other.

The first and second electromagnetic spectral ranges may be different as described elsewhere herein. In some cases, the first portion of the light signals may comprise one or more wavelengths from the visible electromagnetic spectrum. In some cases, the first portion of the light signals may comprise one or more wavelengths from the IR spectrum. In some cases, the first portion of the light signals may comprise one or more wavelengths from the UV spectrum. In some cases, the second portion of the light signals may comprise one or more wavelengths from the visible electromagnetic spectrum. In some cases, the second portion of the light signals may comprise one or more wavelengths from the IR spectrum. In some cases, the second portion of the light signals may comprise one or more wavelengths from the UV spectrum.

The optics assembly may not comprise any focusing device (e.g., an optical aperture such as an objective lens) ahead of the beam splitter (e.g., before the light signals reach the beam splitter). Alternatively, the optics assembly may comprise one or more focusing devices ahead of the beam splitter. The optics assembly may comprise at least about 1, 2, 3, 4, 5, or more focusing devices disposed ahead of the beam splitter. The optics assembly may comprise at most about 5, 4, 3, 2, or 1 focusing devices disposed ahead of the beam splitter.

A focusing device may comprise any lens (e.g., fish-eye, elliptical, conical, and the like), reflector, optic, concentrator, or other device that is capable of reflecting or focusing light. In an example, the focusing device may be a relay lens. The optics assembly may comprise at least about one focusing device (e.g., at least about 1, 2, 3, 4, 5, or more focusing devices) for the imaging sensor. The at least one focusing device may be disposed between the beam splitter and the imaging sensor. The optics assembly may comprise at least one focusing device (e.g., at least about 1, 2, 3, 4, 5, or more focusing devices) for the camera. The at least one focusing device may be disposed between the beam splitter and the camera. In some cases, the optics assembly may comprise at least one focusing device (e.g., at least about 1, 2, 3, 4, 5, or more focusing devices) disposed in the optical path between the imaging sensor and the beam splitter.

In some cases, the imaging sensor may be configured to generate a first set of imaging data from the first portion of the light signals, and the camera may be configured to generate a second set of imaging data from the second portion of the light signals. The first set of imaging data and the second set of imaging data may be the same. In an example, the first and second set of imaging data may be the same in order to confirm validity of the collected imaging data. Alternatively, the first and second set of imaging data may be different, e.g., may represent different spectral features of the coral. The first set of imaging data may complement the second set of imaging data. In an example, a visible imaging sensor may be used for coral bleaching while an IR or UV imaging sensor may be used for coral fluorescence.

The plurality of sensors further comprise sensors for collecting a plurality of data related to coral environmental conditions. The trained algorithms analyze the plurality of data to determine the quality of coral environmental conditions. The plurality of coral environmental conditions are then adjusted, by the trained algorithms, to promote an optimum plurality of coral environmental growth conditions for coral growth, coral health, or coral resiliency. The plurality of environmental conditions may comprise pH or salinity of the coral tank water in which the coral are submerged, water flow rates, light levels, water temperature, and the like described elsewhere herein.

The plurality of sensors further comprises sensors for collecting a plurality of data related to coral environmental conditions. The trained algorithms analyze the plurality of data to determine the quality of coral environmental conditions. The plurality of environmental conditions are then adjusted, by the trained algorithms, to promote an optimum plurality of environmental conditions for coral growth, coral health, or coral resiliency. The plurality of environmental conditions comprise a plurality of levels of minerals in the coral tank water in which the corals are submerged. The plurality of levels of minerals comprise calcium, phosphate, nitrogen, nitrate, nitrite, dissolved oxygen levels, and the like described elsewhere herein.

### ECoFaB Trained Algorithms

The disclosure provides methods of processing a plurality of coral growth, health, or resiliency features at a plurality of wavelengths and processing a plurality of coral environmental features for use in analyzing, by the trained algorithms, coral growth, coral health, or coral resiliency. Further, by assessment of coral growth, coral health, or coral resiliency, the trained algorithms may automatically adjust coral environmental features to optimize coral growth, coral health, or coral resiliency. The trained algorithms may include computer vision models described elsewhere herein.

In some embodiments, the trained algorithm applies a machine learning-based classifier on the plurality of coral health features and the plurality of coral environmental features to determine coral growth, coral health, or coral resiliency.

Examples of machine learning-based classifiers may comprise a regression-based learning algorithm, linear or non-linear algorithms, feed-forward neural network, generative adversarial network (GAN), deep residual networks, or region-based convolutional neural network (MaskRCNN). The machine learning-based classifiers may be, for example, unsupervised learning classifiers, supervised learning classifiers, reinforcement learning classifiers, or combinations thereof. An unsupervised learning classifier may be, for example, clustering, hierarchical clustering, k-means, mixture models, DBSCAN, OPTICS algorithm, anomaly detection, local outlier factor, neural networks, autoencoders, deep belief nets, hebbian learning, generative adversarial networks, self-organizing map, expectation-maximization algorithm (EM), method of moments, blind signal separation techniques, principal component analysis, independent component analysis, non-negative matrix factorization, singular value decomposition, or combinations thereof.

An supervised learning classifier may be, for example, support vector machines, linear regression, logistic regression, linear discriminant analysis, decision trees, k-nearest neighbor algorithm, neural networks, similarity learning, or a combination thereof. In some embodiments, the machine learning-based classifier may comprise a deep neural network (DNN). In some embodiments, the MaskRCNN may be, for example, U-Net, ImageNet, LeNet-5, AlexNet, ZFNet, GoogleNet, VGGNet, ResNet18 or ResNet, etc. Other neural networks may be, for example, deep feed forward neural network, recurrent neural network, LSTM (Long Short Term Memory), GRU (Gated Recurrent Unit), Auto Encoder, variational autoencoder, adversarial autoencoder, denoising auto encoder, sparse auto encoder, boltzmann machine, RBM (Restricted BM), deep belief network, generative adversarial network (GAN), deep residual network, capsule network, or attention/transformer networks, etc.

In some cases, the machine learning-based classifier may be trained using a plurality of data obtained from at least about 1 coral event, 2 coral events, 3 coral events, 4 coral events, 5 coral events, 6 coral events, 7 coral events, 8 coral events, 9 coral events, 10 coral events, 15 coral events, 20 coral events, 25 coral events, 50 coral events, 100 coral events, 500 coral events, 1000 coral events, 10000 coral events, or more. The machine learning-based classifier may be trained using a plurality of images obtained from at most about 10000 coral events, 1000 coral events, 500 coral events, 100 coral events, 50 coral events, 25 coral events, 20 coral events, 15 coral events, 10 coral events, 9 coral events, 8 coral events, 7 coral events, 6 coral events, 5 coral events, 4 coral events, 3 coral events, 2 coral events, or less. The machine learning-based classifier may be trained using a plurality of data obtained from at least about 1 coral event to 10000 coral events, 1 coral event to 1000 coral events, 1 coral event to 100 coral events, 1 coral event to 50 coral events, 1 coral event to 25 coral events, 1 coral event to 20 coral events, 1 coral event to 15 coral events, 1 coral event to 10 coral events, 1 coral event to 9 coral events, 1 coral event to 8 coral events, 1 coral event to 7 coral events, 1 coral event to 6 coral events, 1 coral event to 5 coral events, 5 coral events to 10000 coral events, 5 coral events to 1000 coral events, 5 coral events to 100 coral events, 5 coral events to 50 coral events, 5 coral events to 25 coral events, 5 coral events to 20 coral events, 5 coral events to 15 coral events, 5 coral events to 10 coral events, 5 coral events to 9 coral events, 5 coral events to 8 coral events, 5 coral events to 7 coral events, 5 coral events to 6 coral events, 10 coral events to 10000 coral events, 10 coral events to 1000 coral events, 10 coral events to 100 coral events, 10 coral events to 50 coral events, 10 coral events to 25 coral events, 10 coral events to 20 coral events, or 10 coral events to 15 coral events.

In some cases, the machine learning-based classifier may be written in a classification framework. The classification framework may be, for example, PyTorch, BigDL, Caffe, Chainer, Deeplearning4j, Dlib, Intel Data Analytics Acceleration Library, Intel Math Kernel Library, Keras, MATLAB+Deep Learning Toolbox, Microsoft Cognitive Toolkit, Apache MXNet, Neural Designer, OpenNN, Palid ML, Apache SINGA, TensorFlow, Theano, Torch, or Wolfram Mathematica, etc.

In some cases, the machine learning-based classifier may comprise a variety of parameters. The variety of parameters may be, for example, learning rate, minibatch size, number of epochs to train for, momentum, learning weight decay, or neural network layers etc.

In some cases, the learning rate may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some cases, the learning rate may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less. In some cases, the learning rate may be from about 0.00001 to 0.1, 0.00001 to 0.05, 0.00001 to 0.01, 0.00001 to 0.005, 0.00001 to 0.0001, 0.001 to 0.1, 0.001 to 0.05, 0.001 to 0.01, 0.001 to 0.005, 0.01 to 0.1, or 0.01 to 0.05.

In some cases, the minibatch size may be at least about 16, 32, 64, 128, 256, 512, 1024 or more. In some cases, the minibatch size may be at most about 1024, 512, 256, 128, 64, 32, 16, or less. In some cases, the minibatch size may be from about 16 to 1024, 16 to 512, 16 to 256, 16 to 128, 16 to 64, 16 to 32, 32 to 1024, 32 to 512, 32 to 256, 32 to 128, 32 to 64, 64 to 1024, 64 to 512, 64 to 256, or 64 to 128.

In some cases, the neural network may comprise neural network layers. The neural network may have at least about 1, 2, 3, 4, 5, 6, 7, 8, 9, 10, 15, 20, 50, 100, 200, 500, 1000 or more neural network layers. The neural network may have at most about 1000, 500, 200, 100, 50, 20, 15, 10, 9, 8, 7, 6, 5, 4, 3, 2 or less neural network layers. In some cases, the neural network may have about 1 to 1000, 1 to 500, 1 to 100, 1 to 10, 1 to 5, 1 to 3, 3 to 1000, 3 to 500, 3 to 100, 3 to 10, 3 to 5, 5 to 500, 5 to 100, or 5 to 10 neural network layers.

In some cases, the number of epochs to train for may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more. In some cases, the number of epochs to train for may be at most about 10000, 1000, 500, 250, 200, 150, 100, 95, 90, 85, 80, 75, 70, 65, 60, 55, 50, 45, 40, 35, 30, 25, 19, 18, 17, 16, 15, 14, 13, 12, 11, 10, 9, 8, 7, 6, 5, 4, 3, 2 or less. In some cases, the number of epochs to train for may be from about 1 to 10000, 1 to 1000, 1 to 100, 1 to 25, 1 to 20, 1 to 15, 1 to 10, 1 to 5, 10 to 10000, 10 to 1000, 10 to 100, 10 to 25, 10 to 20, 10 to 15, 10 to 12, 20 to 10000, 20 to 1000, 20 to 100, or 20 to 25.

In some cases, the momentum may be at least about 0.1, 0.2, 0.3, 0.4, 0.5, 0.6, 0.7, 0.8, 0.9 or more. In some cases, the momentum may be at most about 0.9, 0.8, 0.7, 0.6, 0.5, 0.4, 0.3, 0.2, 0.1, or less. In some cases, the momentum may be from about 0.1 to 0.9, 0.1 to 0.8, 0.1 to 0.7, 0.1 to 0.6, 0.1 to 0.5, 0.1 to 0.4, 0.1 to 0.3, 0.1 to 0.2, 0.2 to 0.9, 0.2 to 0.8, 0.2 to 0.7, 0.2 to 0.6, 0.2 to 0.5, 0.2 to 0.4, 0.2 to 0.3, 0.5 to 0.9, 0.5 to 0.8, 0.5 to 0.7, or 0.5 to 0.6.

In some cases, learning weight decay may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some cases, the learning weight decay may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less. In some cases, the learning weight decay may be from about 0.00001 to 0.1, 0.00001 to 0.05, 0.00001 to 0.01, 0.00001 to 0.005, 0.00001 to 0.0001, 0.001 to 0.1, 0.001 to 0.05, 0.001 to 0.01, 0.001 to 0.005, 0.01 to 0.1, or 0.01 to 0.05.

In some cases, the machine learning-based classifier may use a loss function. The loss function may be, for example, regression losses, mean absolute error, mean bias error, hinge loss, adam optimizer and/or cross entropy.

In some cases, the machine learning-based classifier may segment images. The machine learning-based classifier may segment images into categories. In some cases, the machine learning-based classifier may segment images into categories of at least about 1, 2, 3, 4, 5, 6, 7, 8, 9, 10, 15, 20, 25, 50, 100, 150, 200, 250, 300, 350, 400, 450, 500, 1000, 10000, 100000, or more. The machine learning-based classifier may segment images into categories of at most about 100000, 10000, 1000, 500, 450, 400, 350, 300, 250, 200, 150, 100, 50, 25, 20, 15, 10, 9, 8, 7, 6, 5, 4, 3, 2, or less. The machine learning-based classifier may segment images into categories from about 1 to 100000, 1 to 10000, 1 to 1000, 1 to 500, 1 to 450, 1 to 400, 1 to 350, 1 to 300, 1 to 250, 1 to 200, 1 to 150, 1 to 100, 1 to 50, 1 to 25, 1 to 20, 1 to 15, 1 to 10, 1 to 9, 1 to 8, 1 to 7, 1 to 6, 1 to 5, 1 to 4, 1 to 3, 1 to 2, 3 to 100000, 3 to 10000, 3 to 1000, 3 to 500, 3 to 450, 3 to 400, 3 to 350, 3 to 300, 3 to 250, 3 to 200, 3 to 150, 3 to 100, 3 to 50, 3 to 25, 3 to 20, 3 to 15, 3 to 10, 3 to 9, 3 to 8, 3 to 7, 3 to 6, 3 to 5, or 3 to 4.

In some cases, the machine learning-based classifier may comprise a multi-class model. The multi-class model may comprise at least about 2, 3, 4, 5, 6, 7, 8, 9, 10, 15, 20, 25, 30, 35 40, 45, 50, 55, 60, 65, 70, 75, 80, 85, 90, 95, 100, 200, 500, 1000, 5000, 10000, 50000, 100000, or more different coral growth, coral health, or coral resiliency groups. The multi-class model may comprise at most about 100000, 50000, 10000, 5000, 1000, 500, 200, 100, 95, 90, 85, 80, 75, 70, 65, 60, 55, 50, 45, 40, 35, 30, 25, 20, 15, 10, 9, 8, 7, 6, 5, 4, 3, 2, or less different coral growth, coral healthy, or coral resiliency groups. The multi-class model may comprise from about 2 to 100000, 2 to 10000, 2 to 1000, 2 to 100, 2 to 50, 2 to 10, 2 to 9, 2 to 8, 2 to 7, 2 to 6, 2 to 5, 2 to 4, 2 to 3, 3 to 50, 3 to 10, 3 to 9, 3 to 8, 3 to 7, 3 to 6, 3 to 5, 3 to 4, 5 to 50, 3 to 5, 5 to 9, 5 to 8, 5 to 7, 5 to 6, 3 to 5, or 3 to 4 different coral growth, coral health, or coral resiliency groups.

In some cases, the machine learning-based classifier may comprise a multi-class model that may classify a pixel of a coral image. The machine learning-based classifier may classify a pixel of an image into categories of at least about 1, 2, 3, 4, 5, 6, 7, 8, 9, 10, 15, 20, 25, 50, 100, 150, 200, 250, 300, 350, 400, 450, 500, 1000, 10000, 100000, or more. The machine learning-based classifier may classify a pixel of an image into categories of at most about 100000, 10000, 1000, 500, 450, 400, 350, 300, 250, 200, 150, 100, 50, 25, 20, 15, 10, 9, 8, 7, 6, 5, 4, 3, 2, or less. The machine learning-based classifier may classify a pixel of an image into categories from about 1 to 100000, 1 to 10000, 1 to 1000, 1 to 500, 1 to 450, 1 to 400, 1 to 350, 1 to 300, 1 to 250, 1 to 200, 1 to 150, 1 to 100, 1 to 50, 1 to 25, 1 to 20, 1 to 15, 1 to 10, 1 to 9, 1 to 8, 1 to 7, 1 to 6, 1 to 5, 1 to 4, 1 to 3, 1 to 2, 3 to 100000, 3 to 10000, 3 to 1000, 3 to 500, 3 to 450, 3 to 400, 3 to 350, 3 to 300, 3 to 250, 3 to 200, 3 to 150, 3 to 100, 3 to 50, 3 to 25, 3 to 20, 3 to 15, 3 to 10, 3 to 9, 3 to 8, 3 to 7, 3 to 6, 3 to 5, or 3 to 4.

The machine learning-based classifier may classify a pixel of a coral image according to a pre-defined dictionary. The pre-defined dictionary may classify a pixel as a good foreground, bad foreground, and/or background. In some cases, the good foreground may, for example, represent a healthy coral and the bad background may, for example, represent an unhealthy coral. In some cases, the good foreground may, for example, represent a single coral and the bad background may for example, represent two corals. In some cases, the machine learning-based classifier may classify a pixel of an image based on its pixel value and color space/model as described elsewhere herein.

In some cases, the machine learning-based classifier may output image files. The machine learning-based classifier may output a visible image or an overlay of an IR image with a visible image. The machine learning-based classifier may output a visible image or an overlay of an UV image with a visible image. The machine learning-based classifier may output a visible image or an overlay of an IR image and a UV image with a visible image.

In some cases, the machine learning-based classifier may be configured to classify the plurality of coral images at an accuracy of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 996%, 97%, 98%, 99% or more. The machine learning-based classifier may be configured to classify the plurality of coral images at an accuracy of at most 99%, 98%, 97% 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50% or less. The machine learning-based classifier may be configured to classify the plurality of coral images at an accuracy from about 50% to 99%, 50% to 95%, 50% to 90%, 50% to 85%, 50% to 80%, 50% to 75%, 50% to 70%, 50% to 65%, 50% to 60%, 50% to 55%, 60% to 99%, 60% to 95%, 60% to 90%, 60% to 85%, 60% to 80%, 60% to 75%, 60% to 70%, 60% to 65%, 66% to 99%, 66% to 95%, 66% to 90%, 66% to 85%, 66% to 80%, 66% to 75%, 66% to 70%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 59% to 99%.

In some cases, the machine learning-based classifier may utilize a reconstructed phase image to extract features (e.g., coral bleaching, exposed coral skeleton, coral tissue loss, and the like as described elsewhere herein). The features may pertain to the entire coral. The features may pertain to the tentacles of the coral. The features may pertain to the mouth of the coral. The features may pertain to the columella of the coral. The features may pertain to the septa of the coral. The features may pertain to other parts of the coral.

In some cases, the machine learning-based classifier may need to extract and draw relationships between features as conventional statistical techniques may not be sufficient. In some cases, machine learning algorithms may be used in conjunction with conventional statistical techniques. In some cases, conventional statistical techniques may provide the machine learning algorithm with preprocessed features. In some cases, the features may be classified into any number of categories.

In some cases, the machine learning-based classifier may prioritize certain features of coral growth, coral health, or coral resiliency. The machine learning algorithm may prioritize features that may be more relevant for health-dependent phenotypes or morphological changes. The feature may be more relevant for detecting health-dependent phenotypes or morphological changes if the feature is classified more often than another feature. In some cases, the features may be prioritized using a weighting system. In some cases, the features may be prioritized on probability statistics based on the frequency and/or quantity of occurrence of the feature. The machine learning algorithm may prioritize features with the aid of a human and/or computer system.

In some cases, the machine learning-based classifier may prioritize certain features to reduce calculation costs, save processing power, save processing time, increase reliability, or decrease random access memory usage, etc.

In some cases, any number of features may be classified by the machine learning-based classifier. The machine learning-based classifier may classify at least about 3, 4, 5, 6, 7, 8, 9, 10, 15, 20, 25, 50, 100, 500, 1000, 10000 or more features. In some cases, the plurality of features may include between about 3 features to about 10000 features. In some cases, the plurality of features may include between about 10 features to about 1000 features. In some cases, the plurality of features may include between about 50 features to about 500 features.

In some cases, the machine learning algorithm may prioritize certain features of coral growth, coral health, or coral resiliency. The machine learning algorithm may prioritize features that may be more relevant for determining the health of one or more corals. The feature may be more relevant for determining the health of one or more corals if the feature is classified more often than another feature. In some cases, the features may be prioritized using a weighting system. In some cases, the features may be prioritized on probability statistics based on the frequency and/or quantity of occurrence of the feature. The machine learning algorithm may prioritize features with the aid of a human and/or computer system. In some cases, one or more of the features may be used with machine learning or conventional statistical techniques to determine if a segment is likely to contain artifacts.

In some cases, processing the plurality of images of the plurality of coral tanks, coral trays, coral plugs, or coral may further comprise size filtering, background subtraction, elimination of imaging artifacts, cropping, magnification, resizing, rescaling, and color, contrast, brightness adjustment, or object segmentation.

### Computer System

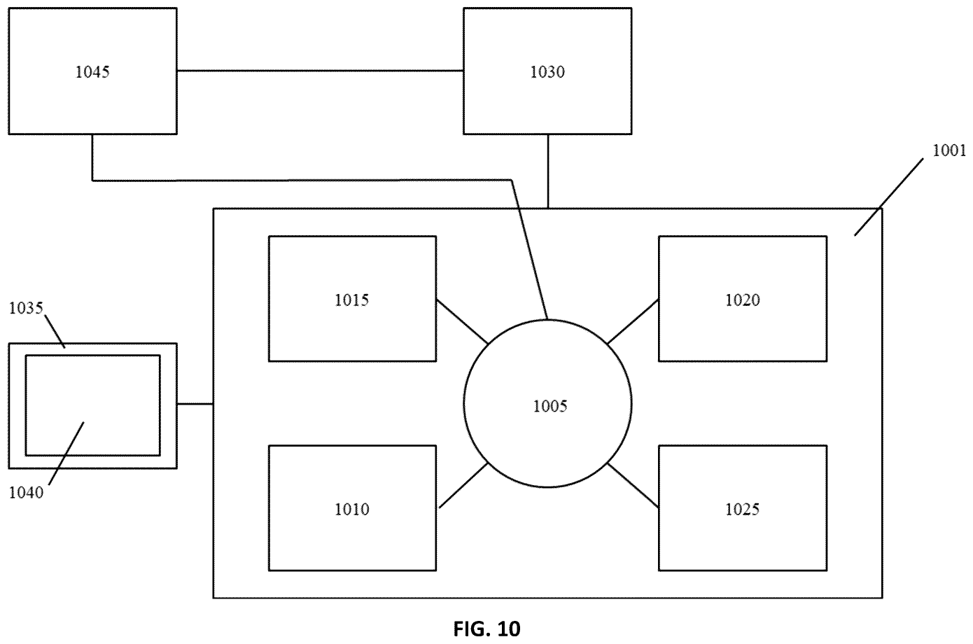

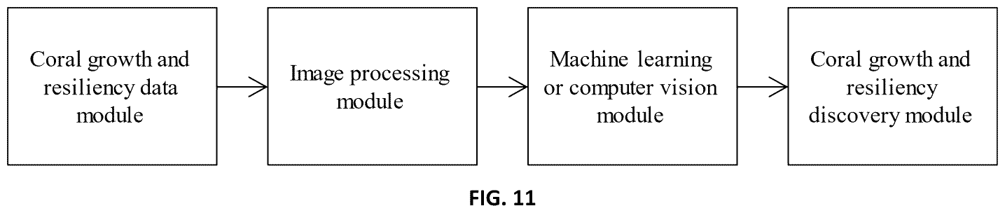

The present disclosure provides computer systems that are programmed to implement methods of the disclosure. FIG. 10 shows a computer system 1001 that is programmed or otherwise configured to implement methods provided herein. The computer system 1001 can regulate various aspects of the present disclosure, such as, for example (a) obtaining one or more images of the one or more corals and (b) applying a machine learning-based classifier comprising a multi-class model on the one or more images to determine a resiliency of the one or more corals based at least on a plurality of coral health features and a plurality of coral environmental features.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet.

The network 1030 in some cases is a telecommunication and/or data network. The network 1030 can include one or more computer servers, which can enable distributed computing, such as cloud computing. For example, one or more computer servers may enable cloud computing over the network 1030 (“the cloud”) to perform various aspects of analysis, calculation, and generation of the present disclosure, such as, for example (a) obtaining one or more images of the one or more corals and (b) applying a machine learning-based classifier comprising a multi-class model on the one or more images to determine a resiliency of the one or more corals based at least on a plurality of coral health features and a plurality of coral environmental features.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems 1045 through the network 1030. For instance, the computer system 1001 can communicate with a remote computer system associated with another coral farm. Examples of remote computer systems include personal computers (e.g., portable PC), slate or tablet PC's (e.g., Apple® iPad, Samsung Galaxy Tab), telephones, Smart phones (e.g., Apple® iphone, Android-enabled device, Blackberry®), or personal digital assistants.

Methods as described herein can be implemented by way of machine (e.g., computer processor) executable code stored on an electronic storage location of the computer system 1001, such as, for example, on the memory 1010 or electronic storage unit 1015. The machine executable- or machine-readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010. The user can access the computer system 1001 via the network 1030.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040. Examples of user interfaces (UIs) include, without limitation, a graphical user interface (GUI) and web-based user interface. For example, the computer system can include a web-based dashboard (e.g., a GUI) configured to display, for example, a high-level architecture comprising plumbing features for controlling injection and extraction of water and nutrients to a user.

Methods and systems of the present disclosure can be implemented by way of one or more algorithms. An algorithm can be implemented by way of software upon execution by the central processing unit 1005. The algorithm can, for example, (a) obtain one or more images of the one or more corals and (b) apply a machine learning-based classifier comprising a multi-class model on the one or more images to determine a resiliency of the one or more corals based at least on a plurality of coral health features and a plurality of coral environmental features.

The present disclosure is not limited to the algorithms disclosed herein. It should be appreciated that other algorithms compatible for use with the described embodiments may be contemplated.

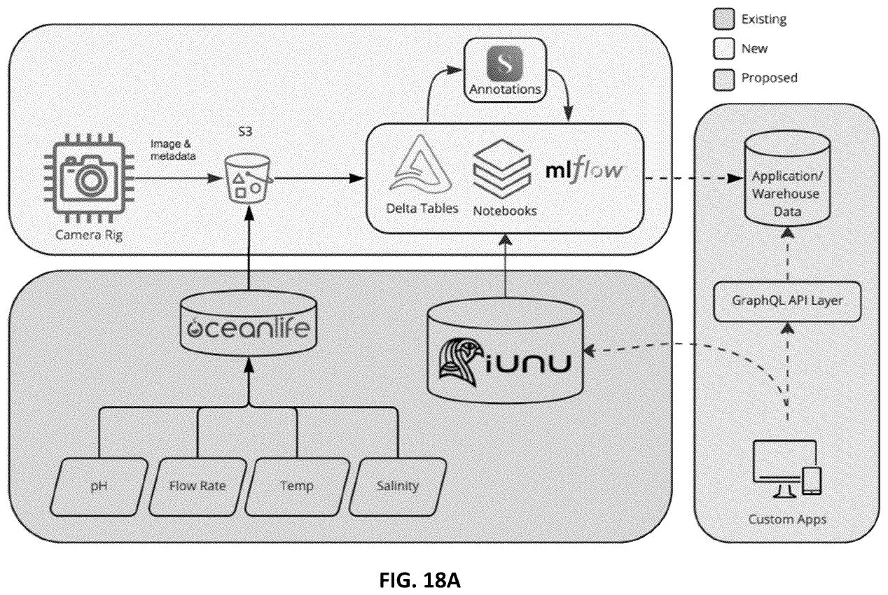

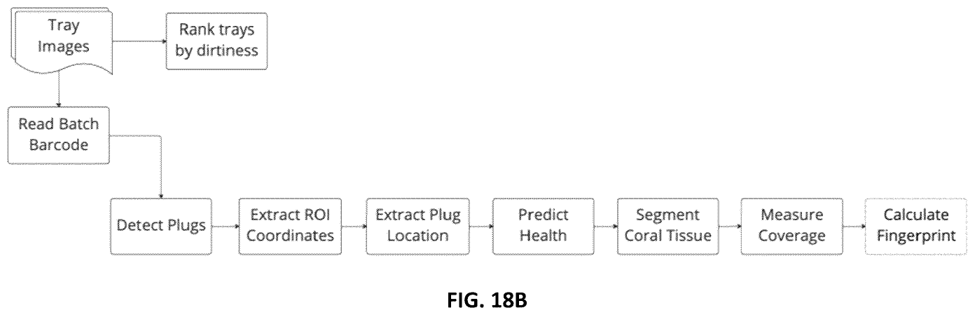

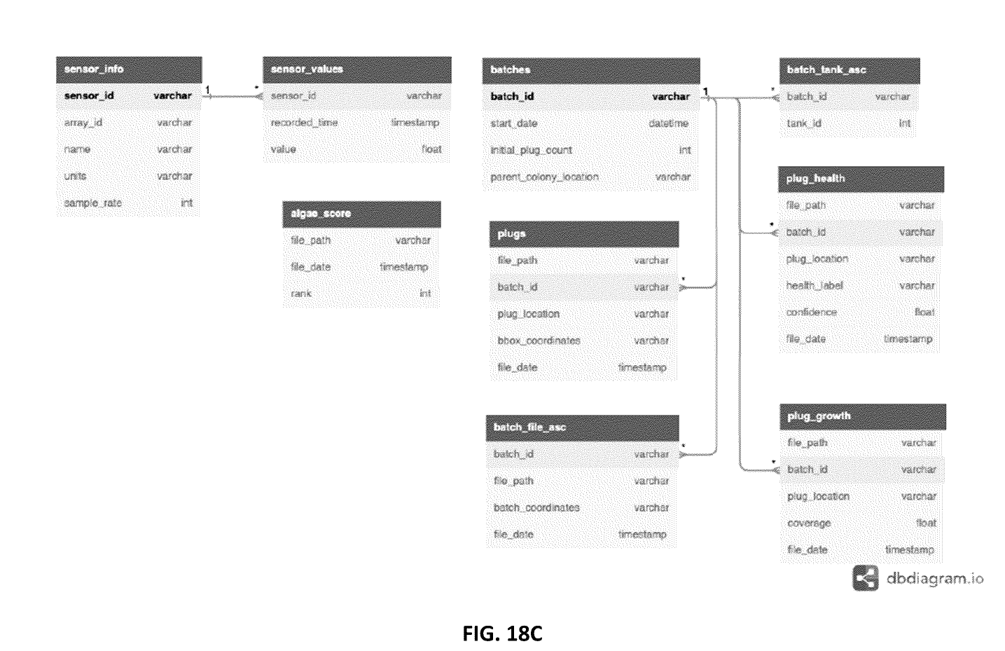

In another embodiment, FIG. 18A depicts a high-level data architecture that may be configured to implement methods of the present disclosure. The data architecture may be configured to operate with the environmental coral farm booth (ECoFaB) system. The data architecture may receive images of coral tanks, coral trays, coral plugs, or coral from the ECoFaB system described elsewhere herein. The data architecture may receive data from a plurality of sensors described elsewhere herein. Images and data may be transferred and stored in cloud object storage e.g., Amazon S3 cloud object storage. Images and sensor data may then be used by trained machine learning (ML) algorithms described elsewhere herein e.g., computer vision models. An image and sensor data pipeline may include an extract, transform, and load (ETL) pipeline such as Databricks® Data Lakes. The ETL pipeline may include operations performed by Databricks Delta Table, Databricks® notebooks, Databricks® MLflow, and Databricks® Annotations. As shown in FIG. 18B, operations may include ranking coral trays by cleanliness or dirtiness (e.g., algae coverage), reading barcodes on coral trays, detecting or identifying coral plugs, determining or extracting regions of interest (ROI) of coral, determining or extracting coral plug locations, determining or predicting coral health or coral resiliency, segmenting coral, determining or predicting coral growth (e.g., measuring coverage), or generating coral fingerprints. Output data of the ETL pipeline can be stored using a database structure shown in FIG. 18C. The database structure may include, for example, sensor information, sensor values, algae scores, batches (e.g., coral tanks and files), coral plugs, coral plug health, or coral plug growth.

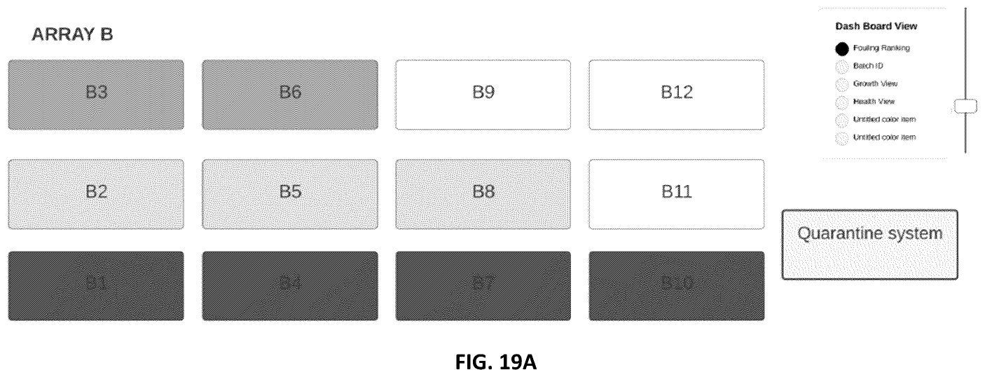

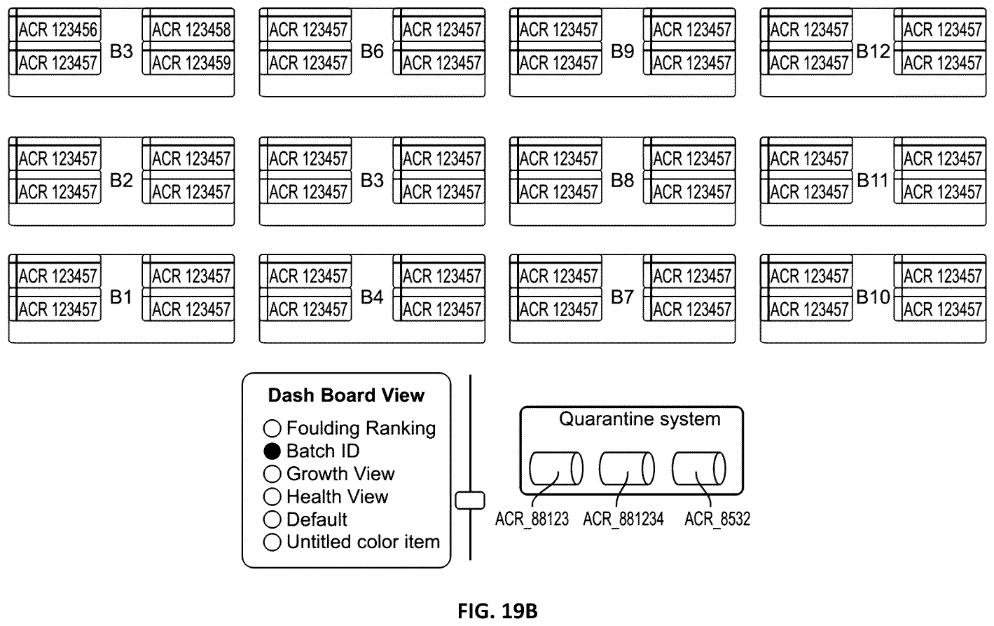

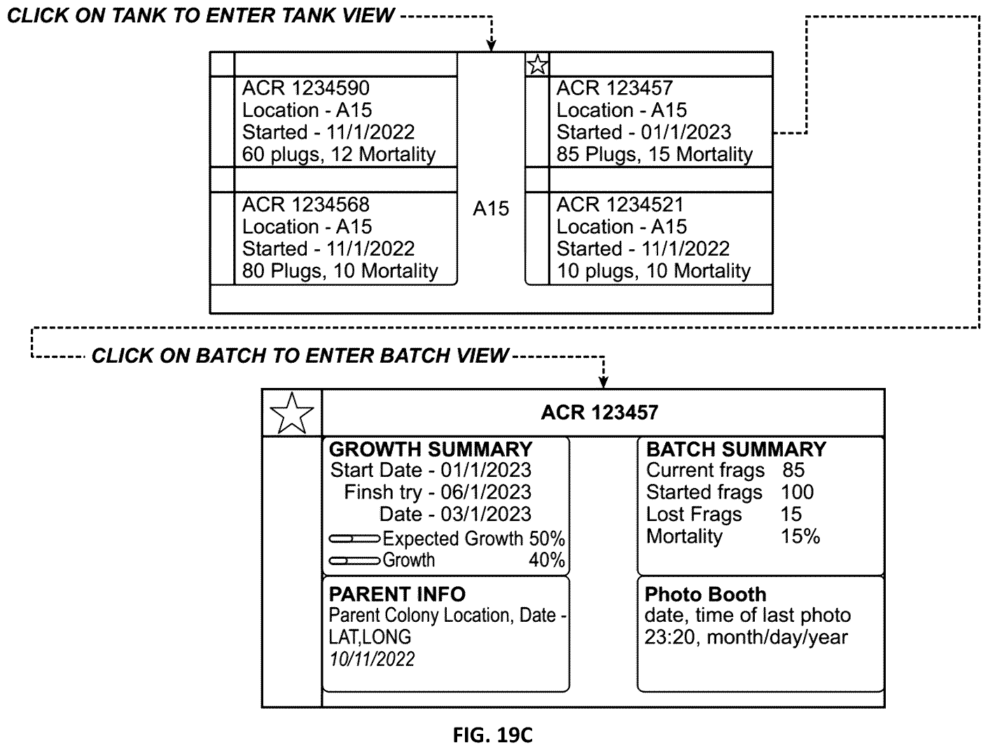

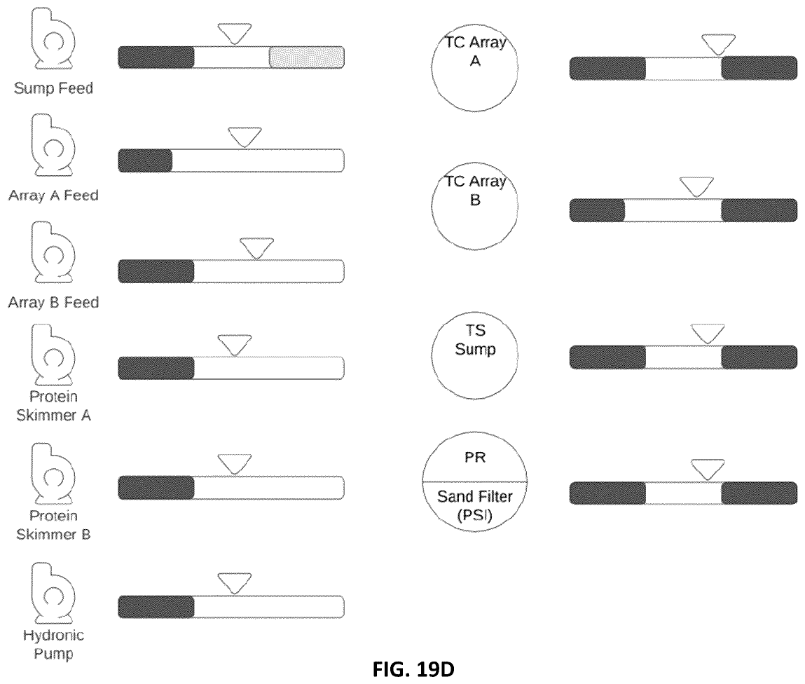

Outputs of the ETL pipeline can be transferred to an application or data warehouse. Application programming interfaces (API) may be configured to access, manipulate, or display data in the application or data warehouse. APIs can communicate with the application or data warehouse via an API layer such as GraphQL API Layer. APIs may be configured to allow users or operators to access, manipulate, or display data in the application or data warehouse via graphical user interfaces (GUI), dashboards, or custom applications. For example, FIGS. 19A-19D depict dashboards that may be generated and configured for users or operators associated with the ECoFaB system or rig. FIG. 19A depicts a dashboard tank view capturing information at the coral tank level to, for example, prioritize which coral tanks require cleaning using a color coding scheme. Each element, batch ID B1-B12, in the array may correspond to a coral tank having coral trays, coral plugs, or coral and can display, for example, cleanliness/fouling ranking (e.g., coral hygiene), batch ID, coral growth, or coral health. FIG. 19B depicts a dashboard coral batch view capturing information at the coral batch level and can display, for example, cleanliness/fouling ranking (e.g., coral hygiene), batch ID, coral growth, or coral health. FIG. 19C depicts a dashboard coral batch view capturing detailed information at the coral batch level and can display, for example, coral growth summary, batch summary, parent coral information, or photobooth ECoFaB information. FIG. 19D depicts a dashboard environmental view for a user or operator to view environmental conditions associated with the ECoFaB system or rig described elsewhere herein. The dashboard may allow a user or operator to visualize coral tank environmental conditions relative to defined thresholds and to alert the user or operator when environmental conditions exceed predetermined thresholds. The dashboard may display real-time data or historical data enabling methods and systems described herein to generate predictions about future outcomes and to diagnose anomalies.

### EXAMPLES

The following illustrative examples are representative of embodiments of the methods an systems described herein and are not meant to be limiting in any way.

### Example 1: Computer Vision Methods

### Data Labeling and Batch Identification

Trained algorithms may include computer vision models to determine, for example, coral growth, coral health, or coral resiliency. Training the algorithms may include labeling images comprising coral tanks, coral trays, coral plugs, or coral. Labeling images may include supervised methods, unsupervised methods, or a combination of both. Training operations may generally include, for example, domain and data exploration, model selection, data labeling, training, testing, or model validation.

Data labels may include, for example, coral-plug, coral-tissue, healthy, unhealthy, clean, intermediate, not-clean, or species. Images labeled as coral-plug can include applying bounding boxes around coral plugs in images of coral tanks, coral trays, coral plugs, or coral. Images labeled as coral-tissue can include applying polygon masks around edges of coral tissue in images of coral tanks, coral trays, coral plugs, or coral. Images labeled as healthy can include labeling as healthy images of coral in images of a coral tanks, coral trays, coral plugs, or coral. Images labeled as unhealthy can include labeling as unhealthy images of coral in images of coral tanks, coral trays, coral plugs, or coral. Unhealthy features may include, for example, coral having bleaching, exposed skeletons, or tissue loss. Images labeled as clean can include labeling as clean images of coral having little or no sign of algae growth in images of coral tanks, coral trays, coral plugs, or coral. Images labeled as intermediate can include labeling as intermediate an images of coral that are neither completely clean nor covered with algae in images of coral tanks, coral trays, coral plugs, or coral. Images labeled as not-clean can include labeling as not-clean images of coral having significant algae growth in images of coral tanks, coral trays, coral plugs, or coral. Images labeled as species can include labeling as species images of coral having a species of coral in images of coral tanks, coral trays, coral plugs, or coral.

For example, supervised training included generating labels shown in in Table 1 for use in training models herein.

TABLE 1

| | Coral- | Coral- | | | | | Not- | |
|---|---|---|---|---|---|---|---|---|
| Label | plug | tissue | Healthy | Unhealthy | Clean | Intermediate | clean | Species |
| Count | 7,510 | 1,359 | 5,208 | 2,086 | 2,303 | 1,473 | 3,612 | 5,668 |

Combinations of supervised and unsupervised learning may include model assisted labeling to label images of coral in images of coral tanks, coral trays, coral plugs, or coral. Operations may include capturing a tray image, detecting coral plugs, predicting health label per coral plug, predicting coral tissue segmentation mask, pre-labeling coral plugs, providing pre-labels to a human operator, approving or disapproving pre-labels, generating updated labels, or training the model based on the updated labels.

Barcodes and the like can be generated and placed on coral trays to aid in identifying batches of coral in images of coral tanks, coral trays, coral plugs, or coral. The barcodes may separate groups of coral plugs belonging to each batch. Coral information may be encoded in the barcodes and include, for example, a species code or a batch number. A pyzbar Python® wrapper around the C zbar barcode reader library may be used to capture or read multiple barcodes in a single image of a coral tray in images of coral tanks, coral trays, coral plugs, or coral. Barcodes can include, for example, quick response (QR) codes, radio-frequency identification (RFID) tags, or any other technology for configuring data encoding.

### Plug Detection

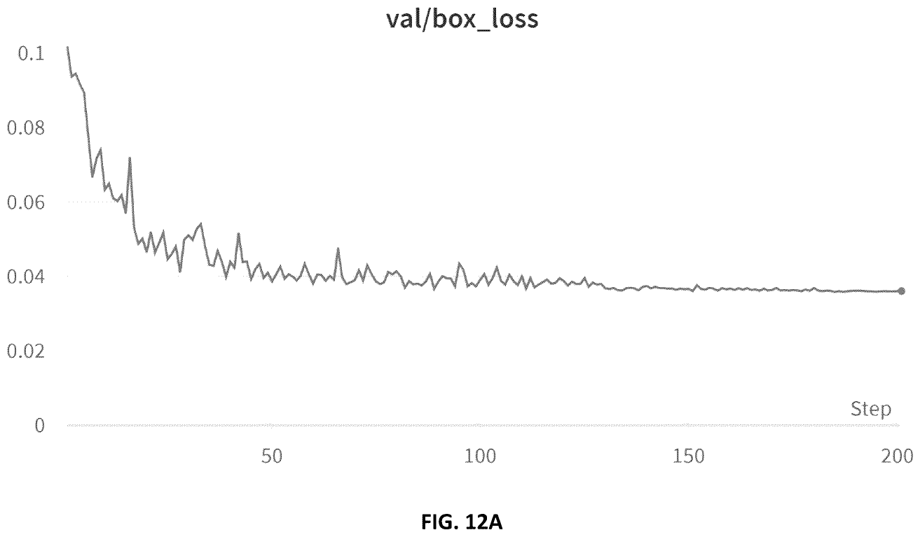

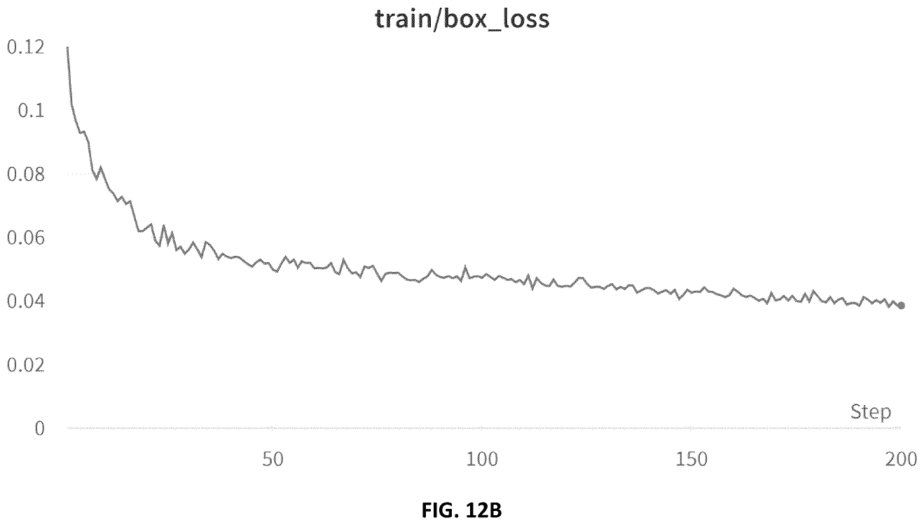

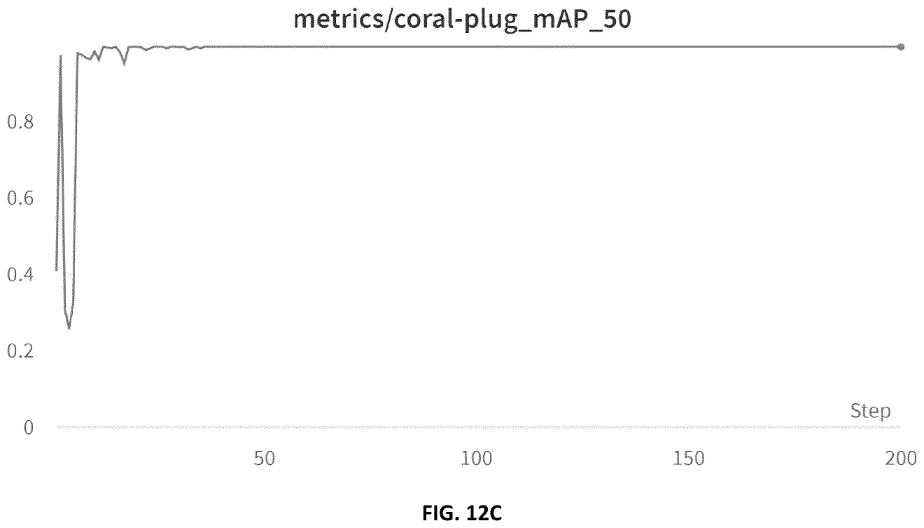

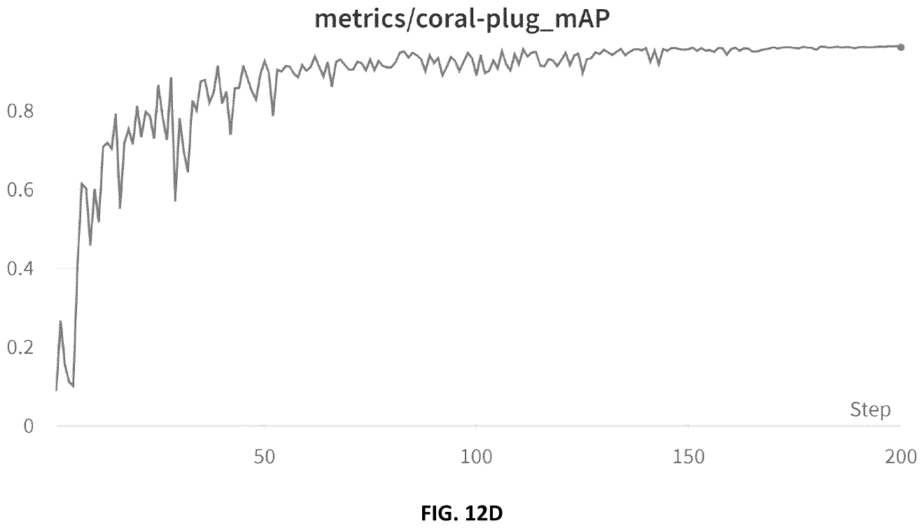

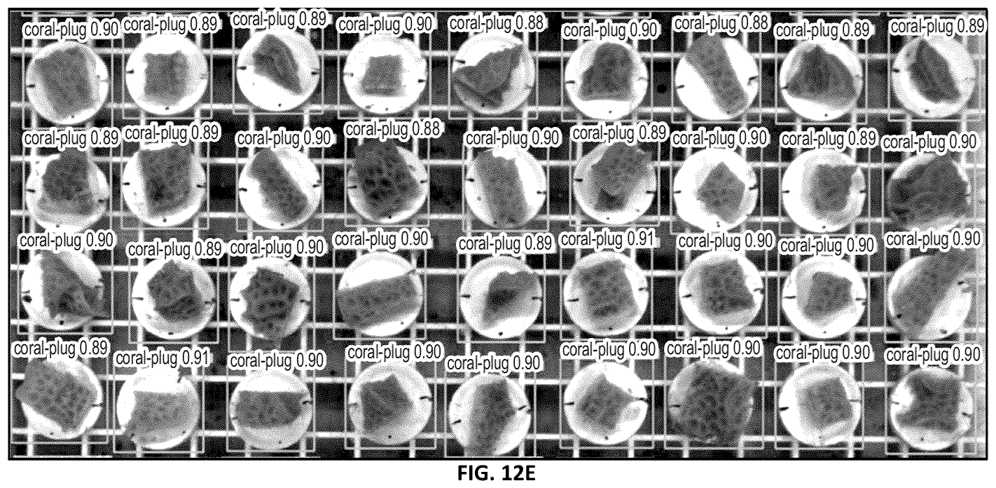

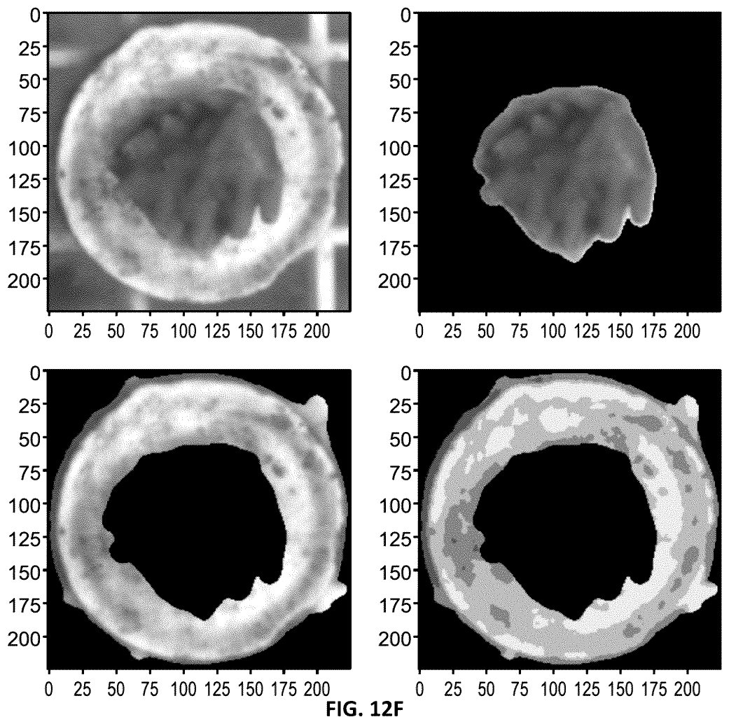

Methods disclosed herein can generate computer vision models to detect and identify coral plugs from, for example, images of coral tanks, coral trays, coral plugs, or coral. The model may be generated using a YOLOv5x6 convolutional neural network architecture. The model can be trained using labeled data. The model may predict and generate bounding boxes around coral plugs in images of coral tanks, coral trays, coral plugs, or coral. For example, 222 tray images having coral plugs were generated. Plugs were labeled to provide labeled data for training, testing, and validation. The data was split into a training set (80%) and a validation set (20%). The validation set was further split into a testing set (20%). Data augmentation or processing included, for example, random rotation, cropping, brightness, and the like. As shown in FIGS. 12A and 12B, training included 200 epochs until the model converged. The evaluation metric and loss function included mean average precision (mAP) and binary cross-entropy with logits loss, respectively. For testing data as shown in FIGS. 12C and 12D, methods disclosed herein achieved a performance of 99.5% mAP with 0.5 intersection over union (IoU) threshold and 95.7% mAP with 0.95 IoU threshold. For validation data as shown in FIGS. 12C and 12D, methods disclosed herein achieved a performance of 99.48% mAP with 0.5 IOU threshold and 96.05% mAP with 0.95 IoU threshold. FIG. 12E illustrates bounding boxes corresponding to detected plugs generated by the computer vision model for 36 images and further includes the certainty that the computer vision model assigns to each bounding box.

FIG. 12F depicts another embodiment of plug detection that may be used to determine coral health, coral growth, or coral resiliency. The method may generate plug surface clustering by inverting a coral mask, extracting a background mask (e.g., comprising a coral plug surface, a coral tray, and a bottom of a coral tank), and determining variations on plug surfaces. The method may use supervised or unsupervised clustering methods. Unsupervised clustering methods may include, for example, mean-shift or k-means to cluster pixels. Pixels can be clustered based on, in part, hue, saturation, and lighting (HSL) values.

### Coral Segmentation

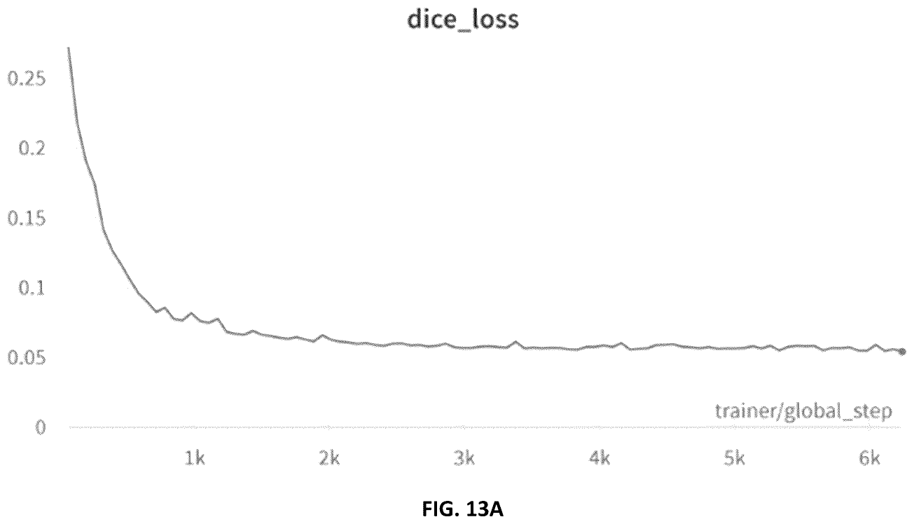

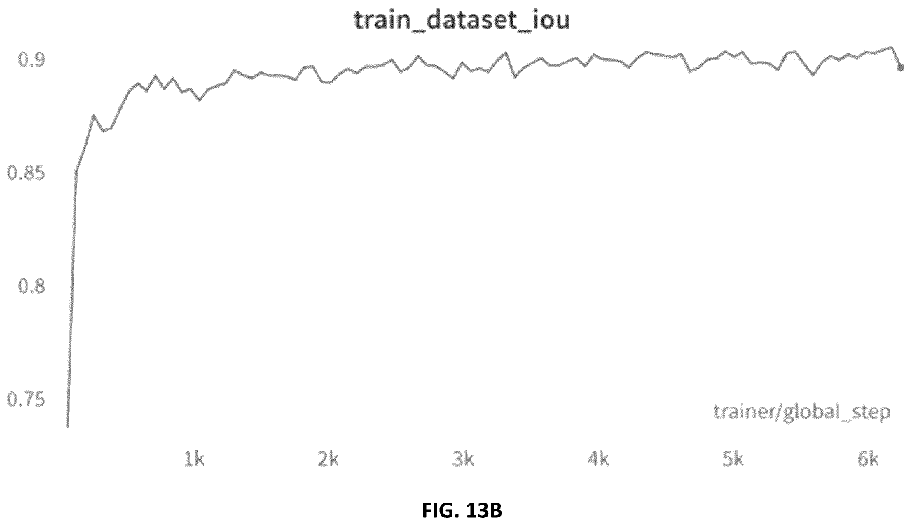

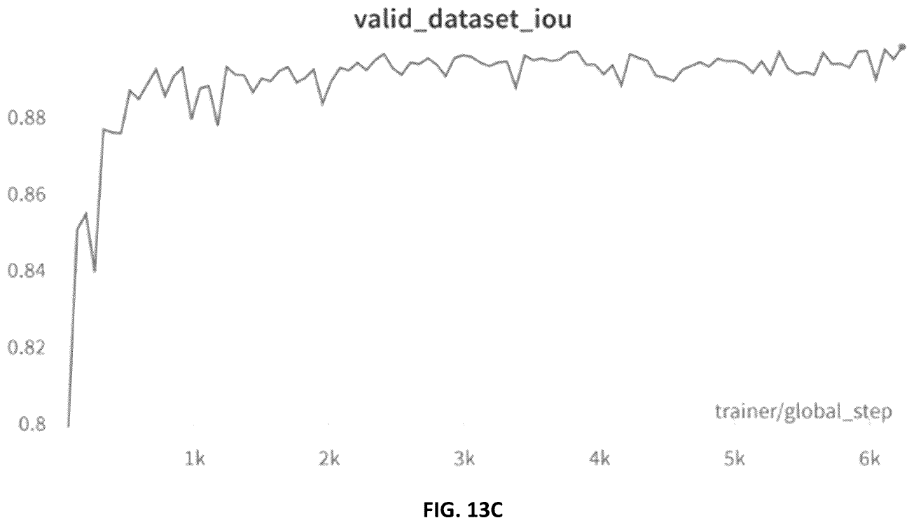

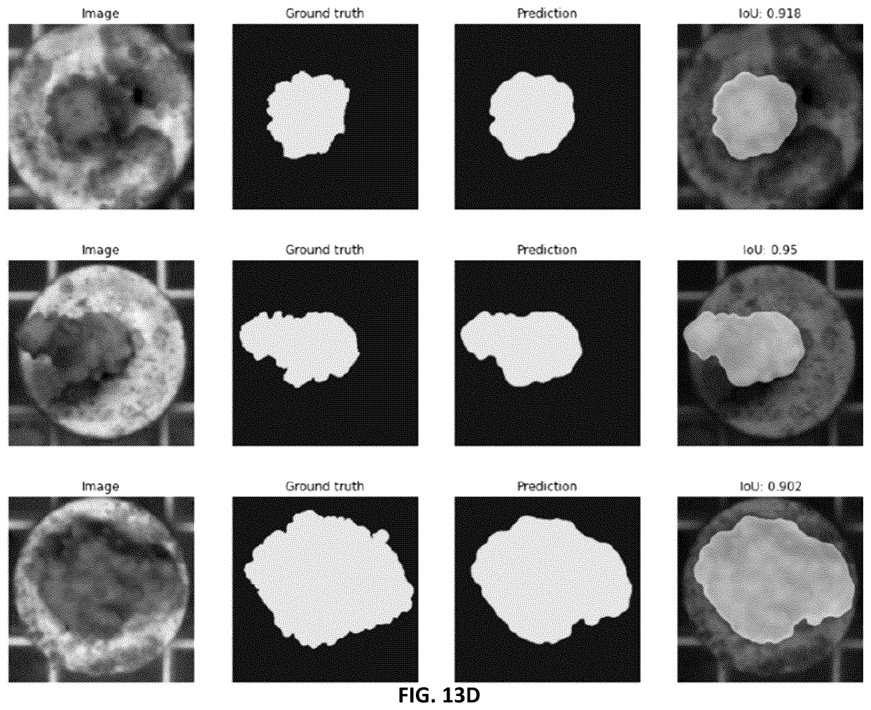

Methods disclosed herein can generate computer vision models to segment out coral tissue from, for example, images of coral tanks, coral trays, coral plugs, or coral. The model may be generated using a Unet++deep neural network. The model can be trained using labeled data. The model may predict and segment coral from coral plugs in images of coral tanks, coral trays, coral plugs, or coral. For example, 1.400 images having coral plugs or coral were generated. Of these, 1,313 coral were labeled to provide labeled data for training, testing, and validation. The data was split into a training set (80%) and a validation set (20%). The validation set was further split into a testing set (10%). Data augmentation or processing included, for example, random rotation, cropping, brightness, and the like. To reduce training time, pre-trained weights from a deep residual network (ResNet) encoder trained on ImageNet© can be used. As shown in FIG. 13A, training included 100 epochs until the model converged. For testing data as shown in FIG. 13B, methods disclosed herein achieved a performance of 89.9% mAP with 0.5 IoU threshold. For validation data as shown in FIG. 13C, methods disclosed herein achieved a performance of 89.8% mAP with 0.5 IoU threshold. FIG. 13D illustrates images, ground truth masks, predictions, and IOU generated by the computer vision model for three images.

### Coral Health

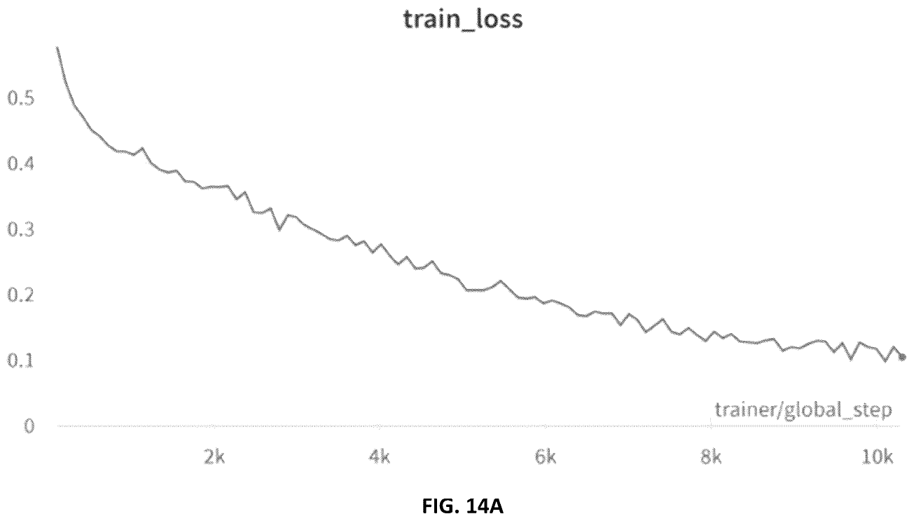

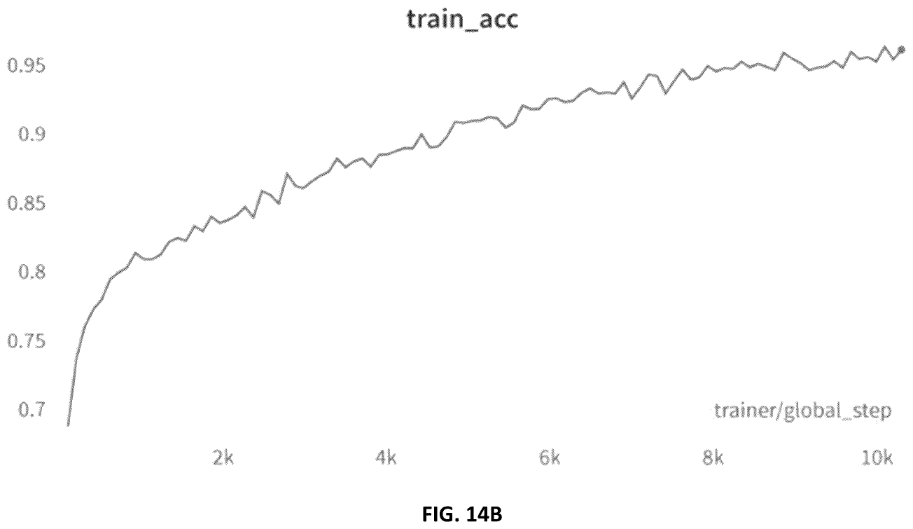

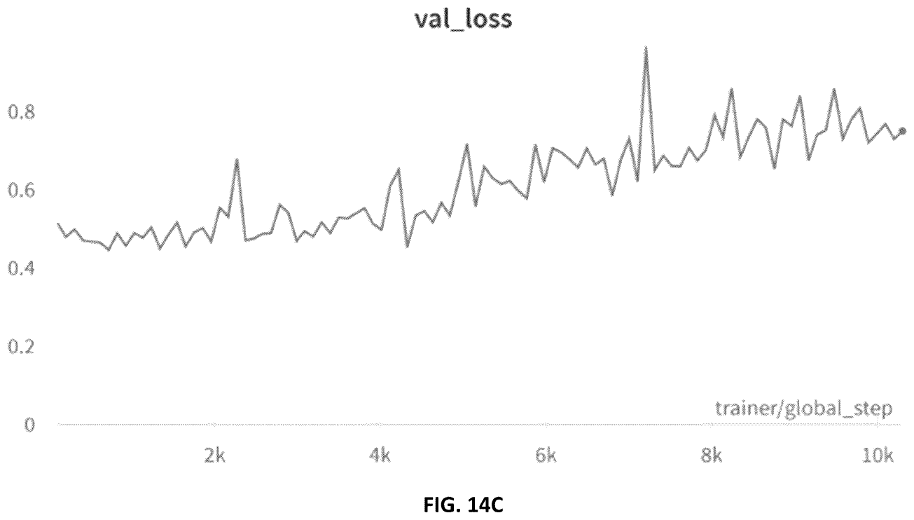

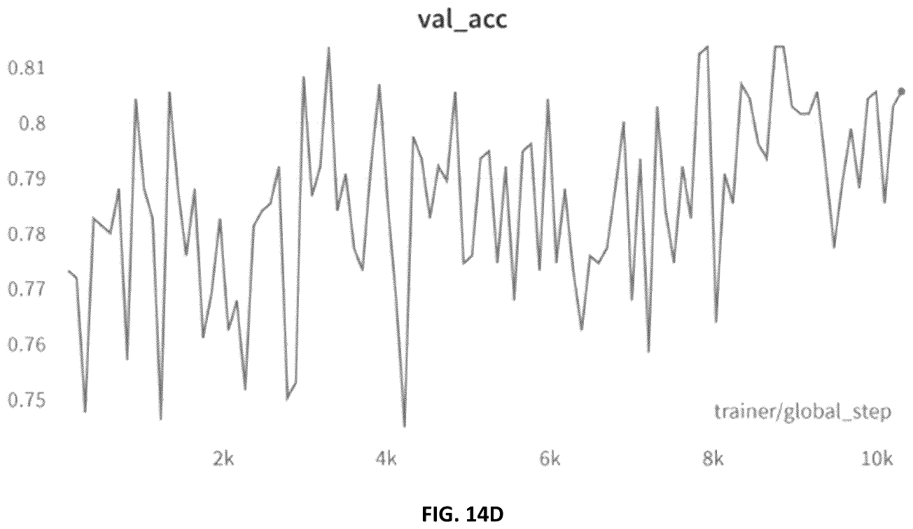

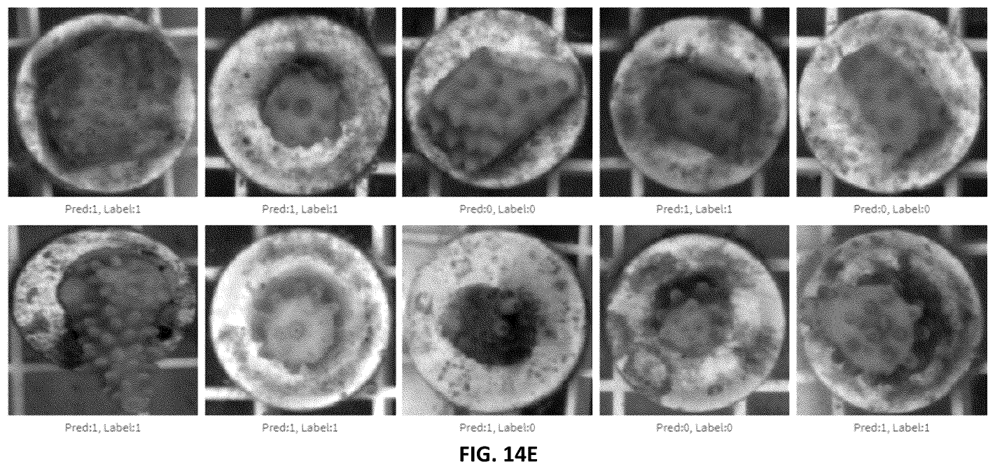

Methods disclosed herein can generate computer vision models to determine health states of coral from, for example, images of coral tanks, coral trays, coral plugs, or coral. The model may predict and classify coral as healthy or unhealthy from images of coral tanks, coral trays, coral plugs, or coral. The binary classifier model can be trained using labeled data. For example, 4,120 images having coral plugs or coral were generated. Healthy and unhealthy coral were equally represented. Coral were labeled to provide labeled data for training, testing, and validation. The data was split into a training set (80%) and a validation set (20%). The validation set was further split into a testing set (10%). Data augmentation or processing included, for example, random rotation, cropping, brightness, and the like. To reduce training time, pre-trained weights from a deep residual network (ResNet) encoder trained on ImageNet© can be used e.g., transfer learning. The evaluation metric and loss function included accuracy and cross-entropy, respectively. For training data as shown in FIGS. 14A and 14B, methods disclosed herein achieved an accuracy of 96.09%. For validation data as shown in FIGS. 14C and 14D, methods disclosed herein achieved an accuracy of 81.38%. For testing data (not shown), methods disclosed herein achieved an accuracy of 86.75%. FIG. 14E compares the predictions of the computer vision model with the labels for 10 images where a 0 represents unhealthy coral and 1 represents healthy coral.

### Coral Growth

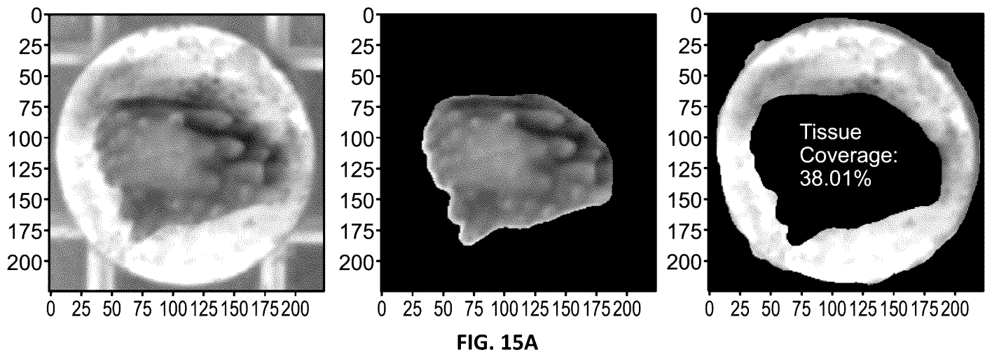

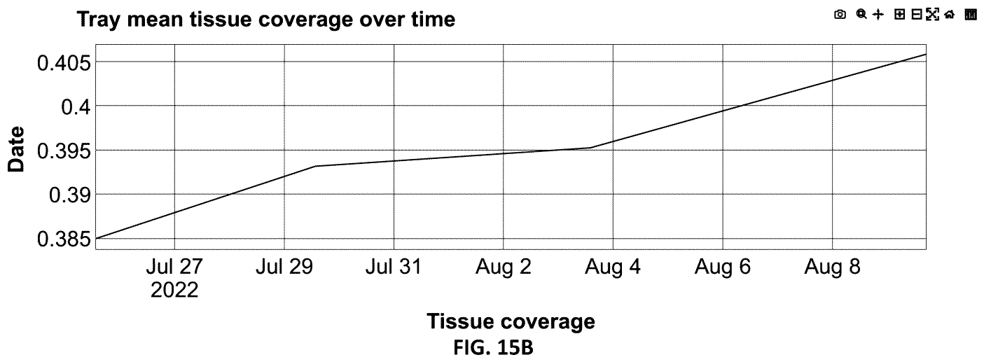

Methods disclosed herein can generate computer vision models to determine coral growth of coral from, for example, images of coral tanks, coral trays, coral plugs, or coral. The model may use as inputs the outputs of associated computer vision models. The outputs may be associated with the outputs of the computer vision plug detection model or the computer vision coral segmentation model described previously herein. For example as shown in FIG. 15A, the computer vision model can determine a pixel area of the coral and subtract a generated pixel area of the coral plug. The computer vision model may perform this operation over different time periods e.g., every 1 day, 1 week, 1 month, or longer. For example as shown in FIG. 15B, the computer vision model determined that coral tissue for a coral increased from about 38.5% tissue coverage to about 40.5% tissue coverage over a period of about 12 days.

### Coral Hygiene or Algae Growth

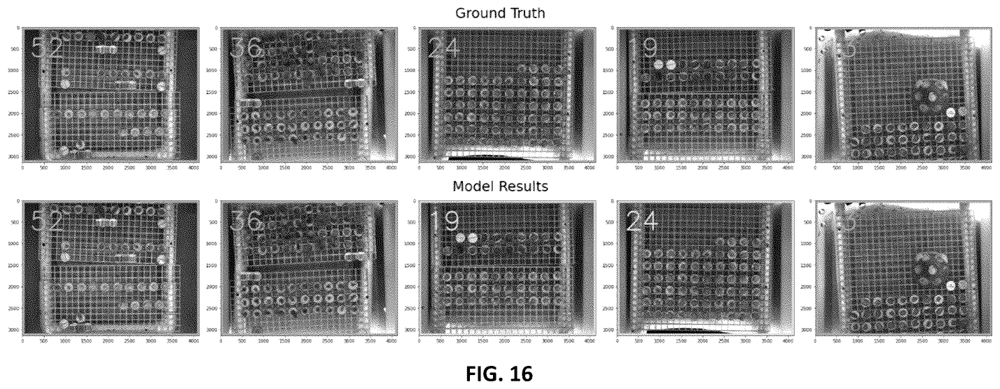

Methods disclosed herein can generate computer vision models to determine or predict coral hygiene or algae growth from, for example, images of coral tanks, coral trays, coral plugs, or coral. The determination or prediction may be used to identify coral tanks, coral trays, coral plugs, or coral requiring cleaning. Various methods can be used. For example, the computer vision model may include a supervised algae segmentation network method to detect algae over a single coral plug surface and also determine algae type. The method may use ground truth masks to label regions of coral plugs having algae. Algae types can be labeled to further train the computer vision model. In another example, a binary classifier may predict whether a coral plug is clean or not clean. In yet another example, the computer vision model may use a siamese network. The siamese network can compare two or more coral trays with one another, e.g., pairwise comparison, to determine or prioritize which coral trays require cleaning of algae. The model can generate a stack-ranked list of coral trays in order of most requiring cleaning to least requiring cleaning. For example, 60 coral tray images having coral plugs or coral were generated. Coral trays were labeled to provide labeled data for training, testing, and validation. The data was split into a training set (80%) and a testing set (20%). Data augmentation or processing included, for example, random rotation, cropping, brightness, and the like. The model generated 1,980 image-pair combinations from the training set and 110 image-pair combinations from the testing set. Training the siamese network included using a convolutional neural network (e.g., a Resnet18 backbone) for 10 epochs until the model converged. Training also included use of a replacement optimization algorithm for stochastic gradient descent for training deep learning models (e.g., Adam optimization with batch size of 8, a scheduled learning rate beginning at 1e-4, a step size of 4, and a gamma of 0.1). The evaluation metric and loss function included accuracy and binary cross-entropy loss, respectively. Methods disclosed herein achieved an accuracy of about 95% on the testing set. FIG. 16 may be used to visualize the accuracy using five images of the plurality of image-pair combinations. Methods disclosed herein ranked coral trays 52, 36, 19, 24, and 13 from lowest to highest requiring cleaning. Labeled images of coral trays ranked coral trays 52, 36, 24, 19, and 13 from lowest to highest requiring cleaning.

### Coral Fingerprinting

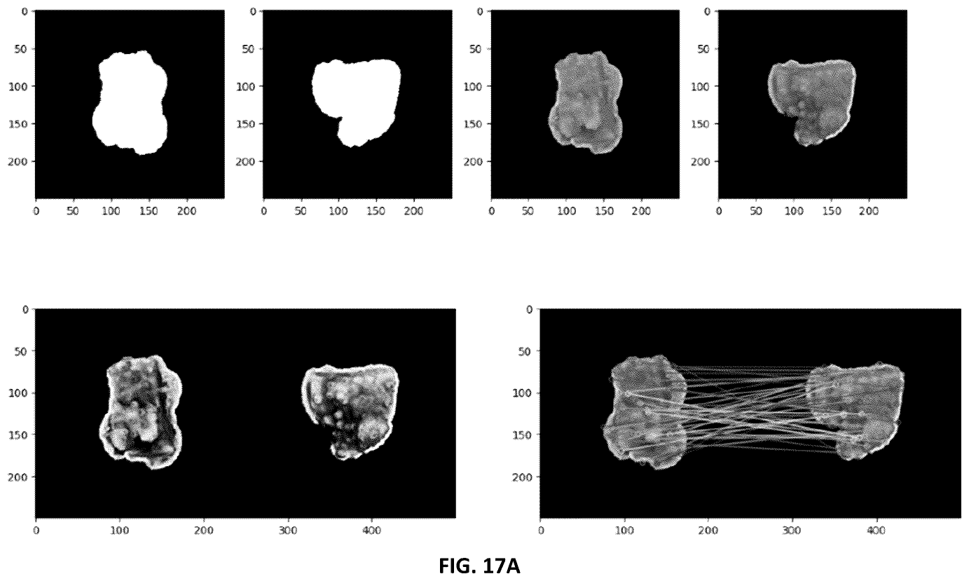

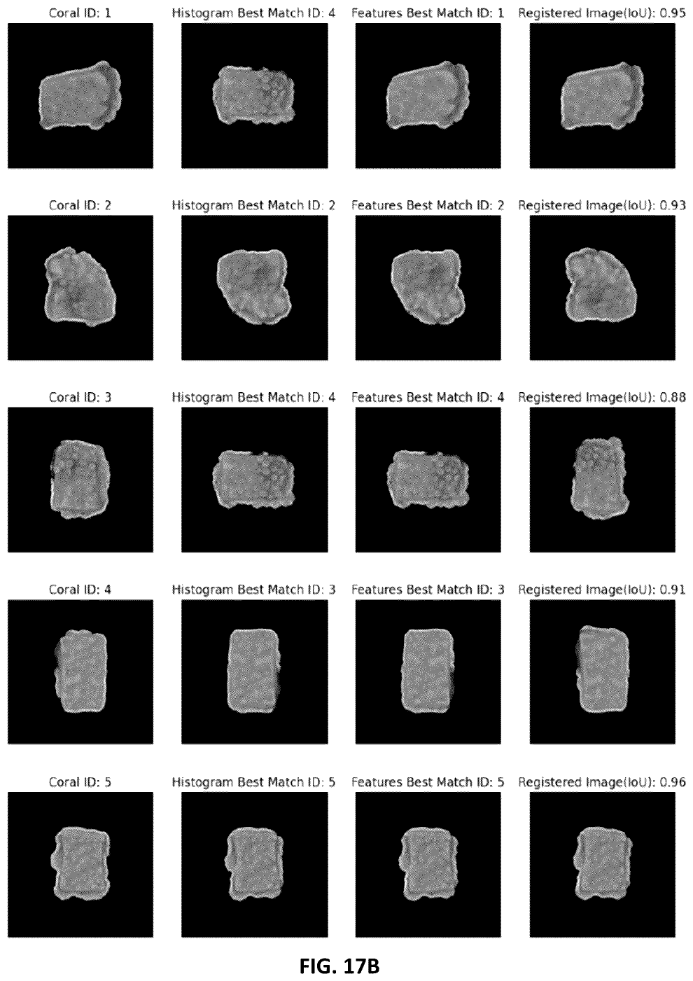

Methods disclosed herein can generate computer vision models to fingerprint coral from, for example, images of coral tanks, coral trays, coral plugs, or coral. Fingerprinting coral can generally mean uniquely identifying coral and tracking coral over time to, for example, determine coral growth, coral health, or coral resiliency. Models can use as inputs coral tray identification data labels (e.g., barcodes described elsewhere herein) or dates of images. Dates of images may be associated with historical dates of coral images or with most recent dates of coral images. As shown in FIG. 17A, fingerprinting algorithm operations may include calculating a widest area of a coral, rotating the coral, comparing to all coral from historical dates (e.g., previous dates of coral images), calculating similarity scores, and determining a best match between the most recent date of a coral image and a previous date of a coral image. The similarity scores may be determined by generating a histogram similarity between two coral images or an IoU between two coral images (e.g., binary masks). Similarity may assess correlation of features (e.g., texture features) between corals. Features of coral can be extracted by a nonlinear, multiscale 2D feature detection and description algorithm such as the KAZE algorithm. Alternatively or additionally, features of coral can be extracted by a fast approximate nearest neighbor search algorithm such as the fast library for approximate nearest neighbors in high dimensional spaces (FLANN) algorithm. For example as shown in FIG. 17B, images of five coral were generated before cleaning and after cleaning (coral numbers 1-5). Methods disclosed herein determined similarity scores before and after cleaning using histogram similarity and feature extraction. Methods disclosed herein achieved IoU performances of 0.95, 0.93, 0.88, 0.91, and 0.96 for coral numbers 1-5, respectively.

### Example 2: Environmental Coral Farm Booth (ECoFaB) System

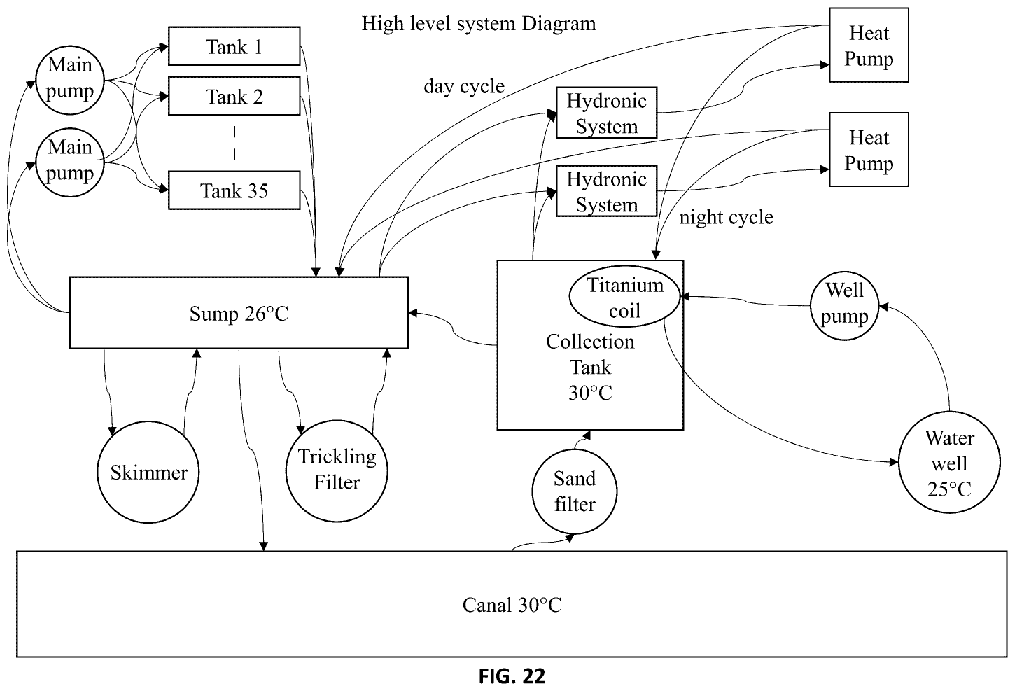

As described elsewhere herein, embodiments of the ECoFaB system (FIGS. 21A-21E) comprise a plurality of plumbing features to operatively function as a coral life support system (LSS) to control injection and extraction of water and nutrients, by the trained algorithm, to and from the plurality of coral tanks, coral trays, coral plugs, coral, or coral microfragments to affect the plurality of coral environmental conditions. The plumbing features further control injection and extraction of chemicals, by the trained algorithms, to maintain optimal environmental conditions in the water tanks for coral growth, coral health, and coral resiliency. For example, FIG. 22 depicts a high-level architecture comprising plumbing features for controlling injection and extraction of water and nutrients for an ECoFaB system. Plumbing features may include, for example, main pumps for flow of water to coral tanks; sumps for return of water from coral tanks; skimmers to remove particles (e.g., algae) from sumps; trickling filters to remove organic matter from sumps, hydronic systems and heat pumps to control water temperatures; collection tanks to control water levels; water wells and well pumps as a source of water; canals to return waters from sumps; and sand/particle filters to remove sand/particle from canals.

Although the description has been described with respect to particular embodiments thereof, these particular embodiments are merely illustrative, and not restrictive. Concepts illustrated in the examples may be applied to other examples and implementations.

While preferred embodiments of the present disclosure have been shown and described herein, it will be obvious to those skilled in the art that such embodiments are provided by way of example only. It is not intended that the present disclosure be limited by the specific examples provided within the specification. While the present disclosure has been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein may be employed in practicing the present disclosure. It is therefore contemplated that the present disclosure shall also cover any such alternatives, modifications, variations, or equivalents. It is intended that the following claims define the scope of the present disclosure and that methods and structures within the scope of these claims and their equivalents be covered thereby.

1.-18. (canceled)

19. A method for determining or predicting coral growth, coral health, or coral resiliency, the method comprising:
(a) obtaining one or more images of one or more corals; and
(b) applying a machine learning-based classifier comprising a multi-class model on the one or more images to determine the coral growth, the coral health, or the coral resiliency of the one or more corals based at least on a plurality of coral growth features, coral health features, coral resiliency features, or coral environmental features.

20. The method of claim 19, wherein the classifier analyzes the one or more images comprising images in the (i) visible electromagnetic (EM) spectrum, (ii) infrared (IR) EM spectrum, or (iii) ultraviolet (UV) EM spectrum.

21. The method of claim 19, wherein (b) comprises using the classifier to analyze the plurality of coral health features, wherein the plurality of coral health features comprises coral bleaching, coral growth, exposed coral skeleton, coral tissue loss, or coral hygiene.

22. The method of claim 19, wherein (b) comprises using the classifier to analyze the plurality of coral environmental features, wherein the plurality of coral environmental features comprises pH or salinity of water in which the one or more corals are submerged.

23. The method of claim 19, wherein (b) comprises using the classifier to analyze the plurality of coral environmental features, wherein the plurality of coral environmental features comprises levels of calcium, phosphate, nitrogen, nitrate, nitrite, or dissolved oxygen in the water in which the one or more corals are submerged.

24. The method of claim 19, further comprising using the classifier to determine or predict the coral growth, the coral health, or the coral resiliency for a likelihood of successful outplanting to an in situ environment.

25. The method of claim 19, further comprising obtaining genetic sequencing of the one more or corals to perform assisted evolution.

26. The method of claim 25, wherein the genetic sequencing is used to train the classifier for performing the assisted evolution of the one or more corals.

27. The method of claim 25, wherein the assisted evolution comprises (i) subjecting the one or more corals to adverse growth or environmental conditions and (ii) obtaining updated genetic sequencing of the subjected one or more corals.

28. The method of claim 25, further comprising using the genetic sequencing to perform the assisted evolution of the one or more corals.

29. The method of claim 19, further comprising adjusting one or more environmental parameters of an environmental apparatus determined by the classifier to have a likelihood of optimizing coral growth conditions.

30. The method of claim 29, wherein the one or more environmental parameters comprise (i) pH or salinity of water in which the one or more corals are submerged or (ii) levels of calcium, phosphate, nitrogen, nitrate, nitrite, or dissolved oxygen in the water.

31. The method of claim 19, further comprising outplanting at least one coral of the one or more corals determined by the classifier to have a likelihood of successful outplanting.

32. The method of claim 20, further comprising using an imaging apparatus to obtain the one or more images of the one or more corals, wherein the imaging apparatus comprises one or more sensors for imaging in (i) the visible EM spectrum, (ii) the IR EM spectrum, or (iii) the UV EM spectrum.

33. The method of claim 19, further comprising processing the one or more images into one or more reconstructed phase images.

34. The method of claim 33, wherein (b) comprises using the classifier to analyze the one or more reconstructed phase images to determine coral health features comprising coral bleaching, coral growth, exposed coral skeleton, coral tissue loss, or coral hygiene.

35. The method of claim 19, wherein the classifier comprises a convolutional neural network (CNN).

36. The method of claim 35, wherein the CNN is trained to obtain (i) a loss function of less than 5% or (ii) an accuracy greater than 95%.

37. The method of claim 35, wherein the CNN obtains an increase in coral tissue coverage of at least about 1% over a predetermined time.

38. The method of claim 35, wherein the CNN prioritizes health-dependent phenotypes or morphological changes based at least on a frequency or a number of occurrences of the health-dependent phenotypes or the morphological changes.
